(19) United States
(12) Patent Application Publication
(10) Pub. No.: US 2023/0226005 A1
(43) Pub. Date: Jul. 20, 2023

(54) Methods of Treating, Ameliorating, Shortening Duration, and/or Reversing Symptoms and/or Complications of a Coronavirus Infection

(71) Applicant: Yale University, New Haven, CT (US)

(72) Inventor: Joseph M. Vinetz, New Haven, CT (US)

(21) Appl. No.: 18/002,477

(22) PCT Filed: Jun. 29, 2021
(86) PCT No.: PCT/US2021/039658

§ 371 Date: Dec. 19, 2022

Related U.S. Application Data
(60) Provisional application No. 63/045,479, filed on Jun. 29, 2020.

(51) Int. Cl. A61K 31/245; A61P 31/14

(52) U.S. Cl. CPC A61K 31/245; A61P 31/14

(57) ABSTRACT

The present disclosure relates, in part, to methods of treating, ameliorating, shortening duration, and/or reversing at least one symptom and/or complication in a human subject with a coronavirus infection with camostat, or a pharmaceutically acceptable salt or solvate thereof. The present disclosure further relates to a method of preventing and/or reducing the occurrence of long COVID, and/or a symptom and/or complication thereof, hospitalization, and/or death in a human subject with a SARS-CoV-2 infection with camostat, or a pharmaceutically acceptable salt or solvate thereof. In certain embodiments, the camostat salt is camostat mesylate.

### CROSS REFERENCE TO RELATED APPLICATIONS

This application claims priority under 35 U.S.C. § 119(e) to U.S. Provisional Patent Application No. 63/045,479, filed Jun. 29, 2020, which application is incorporated herein by reference in its entirety.

### STATEMENT REGARDING FEDERALLY SPONSORED RESEARCH

This invention was made with government support under TR001863 awarded by National Institutes of Health. The government has certain rights in the invention.

### BACKGROUND

SARS-CoV-2, one of a family of human coronaviruses, causes a disease presentation which has now been named COVID-19. Initially identified in Wuhan in December 2019, the virus has subsequently spread throughout the world and was declared a pandemic by the World Health Organization on Mar. 11, 2020. Overall estimation of mortality rates vary from 0.039% to 7%, depending on the efficiency of population-based RT-PCR testing, host demographic, and baseline clinical status. In the U.S., as of Apr. 12, 2020, there were 532,339 confirmed cases and 21,418 deaths, yielding an estimated case-fatality rate of 4% for identified cases.

Patients infected with SARS-CoV can present a range of symptoms. Many infected individuals may be asymptomatic, while others may experience severe respiratory failure, septic shock, and/or multiple organ failure. The most common symptoms are fever, cough, myalgia, and dyspnea, though some patients present with headache, dizziness, nausea, vomiting, and loss of taste and/or smell. Viral pneumonia occurs in severe disease and leads to severe acute respiratory failure.

Entry of the virus into the target cell depends on binding of the spike protein (S), located on the surface of coronaviruses, to a host cellular receptor. SARS-CoV-2 uses the angiotensin converting enzyme II (ACE2) as its cell entry receptor protein to access and infect human cells. Viral entry further requires priming of the S protein by host cellular proteases. This process relies on transmembrane protease serine S1 member 2 (TMPRSS2) expressed on the surface of human epithelial cells of the respiratory and gastrointestinal tracts. Thus, TMPRSS2 priming of the coronavirus S protein is required for ACE2 enabled viral entry to human epithelial cells, and TMPRSS2 has been identified as a target for inhibition of SARS-CoV-2 viral entry.

In vitro studies have suggested inhibition of TMPRSS2 as a potential approach to the prevention of SARS-CoV-2 viral entry, utilizing camostat mesylate as the TMPRSS2 inhibitor. Additionally, in vivo studies using camostat mesylate with a strain of SARS-CoV-1 adapted to causing lethal disease in a mouse model have suggested a mortality reduction from 100% to 30-35%.

The standard of care for COVID-19 is supportive treatment, as there is no approved specific treatment for COVID-19 nor any drug that can be used to treat and/or prevent COVID-19 disease in humans.

Thus, there is a need in the art for a method of treating, ameliorating, shortening duration, and/or reversing symptom(s) of COVID-19 disease and/or complications thereof in a subject, and the present disclosure addresses this need.

### BRIEF SUMMARY

In one aspect, the present disclosure provides a method of treating, ameliorating, shortening duration, and/or reversing at least one symptom and/or complication in a human subject with a coronavirus infection. In certain embodiments, the method comprises administering to the subject a dose of about 645 mg of camostat free base, or an equimolar amount of a pharmaceutically acceptable salt or solvate thereof. In certain embodiments, the camostat salt is camostat mesylate.

In certain embodiments, the coronavirus is at least one of MERS-CoV, SARS-CoV, and/or SARS-CoV-2. In certain embodiments, the coronavirus is SARS-CoV-2. In certain embodiments, the subject suffers from long COVID.

In certain embodiments, the administering reduces and/or prevents progression of the coronavirus infection. In certain embodiments, progression of the coronavirus infection comprises hospitalization and/or death. In certain embodiments, the administering reduces and/or eliminates at least one symptom of the coronavirus infection. In certain embodiments, the administering reduces recovery time for at least one symptom of the coronavirus infection.

In certain embodiments, the at least one symptom is selected from the group consisting of runny nose, congested nose, sinus pressure, sneezing, scratchy or itchy throat, sore or painful throat, swollen throat, difficulty swallowing, teary or water eyes, sore or painful eyes, eyes sensitive to light, difficulty breathing, chest congestion, chest tightness, dry or hacking cough, wet or loose cough, frequent coughing, coughing mucus or phlegm, lack of appetite, gastrointestinal discomfort (i.e., stomach ache), vomiting, diarrhea, headache, head congestion, dizziness, lightheadedness, nausea, excessive sleeping, dyspnea, myalgia, fever, difficulty sleeping, body aches or pains, fatigue, chills or shivering, feeling cold, feeling hot, sweating, discomfort, abnormal, reduced, or eliminated sense of smell (e.g., anosmia), and abnormal, reduced, or eliminated sense of taste (e.g., ageusia).

In certain embodiments, the at least one symptom is anosmia. In certain embodiments, the at least one symptom is ageusia. In certain embodiments, the recovery time is reduced by at least about 1 to about 10 days.

In certain embodiments, the administration to the subject is performed daily. In certain embodiments, the administration to the subject is performed for a period of 7 days. In certain embodiments, the daily administration comprises four equal doses of camostat free base, or a pharmaceutically acceptable salt or solvate thereof. In certain embodiments, each of the four equal doses comprises about 200 mg of camostat mesylate.

### BRIEF DESCRIPTION OF THE FIGURES

The drawings illustrate generally, by way of example, but not by way of limitation, various embodiments of the present application.

FIG. 1 illustrates inhibition of TMPRSS2, and consequently viral entry via the endosomal pathway, via camostat mesylate administration.

FIG. 2A shows the relationship between the concentration of a camostat metabolite [4-(4-guanidinobenzoyloxy)phenylacetic acid, also known as GBPA] in the plasma of a human over time within a 24 hour period with administration of four 200 mg doses of camostat mesylate and different dose intervals ranging from 3 to 6 hours, as determined by pharmacokinetic simulation.

FIG. 2B provides the relationship between average concentration of a camostat metabolite in the plasma of a human and time per day in which the concentration of a camostat metabolite in the plasma of a human is greater than 0.087 μM as a function of camostat mesylate 200 mg QID dose interval, as determined by pharmacokinetic simulation.

FIGS. 3A-3C provide graphs showing the average viral load (log₁₀) as a function of days visited for subjects treated with camostat (solid line) and placebo (dashed line), for the N gene (FIG. 3A), ORF1ab gene (FIG. 3B), and S gene (FIG. 3C).

FIGS. 4A-4C provide graphs showing the average change in viral load (log₁₀) as a function of days visited for subjects treated with camostat (solid line) and placebo (dashed line), for the N gene (FIG. 4A), ORF1ab gene (FIG. 4B), and S gene (FIG. 4C).

FIGS. 5A-5C provide graphs showing least squares mean of viral load (log₁₀) as a function of days visited for subjects treated with camostat (solid line) and placebo (dashed line), for the N gene (FIG. 5A), S gene (FIG. 5B), and ORF1ab (FIG. 5C).

FIGS. 6A-6C provide graphs showing least squares mean of severity score as a function of days visited for subjects treated with camostat (solid line) and placebo (dashed line) using ANCOVA (FIG. 6A), Fitzmaurice (FIG. 6B), and negative binomial (FIG. 6C) models.

FIG. 7 provides a graph showing the least squares mean of change in frequency score as a function of days visited for subjects treated with camostat (solid line) and placebo (dashed line) using the Fitzmaurice model.

FIGS. 8A-8G provide graphs showing the least squares mean of the symptom score using the Fitzmaurice model for nose symptoms (FIG. 8A), throat symptoms (FIG. 8B), eye symptoms (FIG. 8C), chest symptoms (FIG. 8D), gastrointestinal symptoms (FIG. 8E), body symptoms (FIG. 8F), and smell/taste symptoms (FIG. 8G).

### DETAILED DESCRIPTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

The term “ageusia” as used herein refers to a taste disorder characterized by the loss, decrease, and/or absence of the sense of taste.

A disease or disorder is “alleviated” or “ameliorated” if the severity or frequency of at least one sign or symptom of the disease or disorder experienced by a patient is reduced.

As used herein, the term “ALT” refers to the amount of alanine aminotransferase in the blood of a subject.

The term “anosmia” as used herein refers to a partial or complete loss of the sense of smell, which may be temporary or permanent in duration.

As used herein, the term “AST” refers to the amount of aspartate aminotransferase in the blood of a subject.

As used herein, the term “BUN” refers to the amount of urea nitrogen in the blood of a subject.

As used herein, the term “Ct” or “cycle threshold” refers to the number of cycles of the polymerase chain reaction required for a fluorescence detection signal to exceed the threshold of the background in RT-PCR.

The term “complementary” as used herein refers to the broad concept of subunit sequence complementarity between two nucleic acids, e.g., two DNA molecules. When a nucleotide position in both of the molecules is occupied by nucleotides normally capable of base pairing with each other, the nucleic acids are considered to be complementary to each other at this position. Thus, two nucleic acids are substantially complementary to each other when at least 50%, preferably at least about 60% and more preferably at least about 80% of corresponding positions in each of the molecules are occupied by nucleotides which normally base pair with each other (e.g., A:T and G:C nucleotide pairs).

The term “COVID” or “COVID-19” as used herein refers to the Coronavirus disease 2019, a contagious disease caused by severe accurate respiratory syndrome coronavirus 2 (SARS-CoV-2).

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “equimolar” refers to a quantitative comparison of the amount two chemical substances with regard to the number of moles, wherein the two chemical substances have the same number of moles, but not necessarily the same mass due to differences in the molar mass of the two chemical substances. For example, an approximately equimolar mixture of NaCl and KCl may comprise 58.4 g of NaCl and 74.6 g of KCl, as the molar mass of NaCl and KCl are 58.443 g/mol and 74.551 g/mol, respectively.

As used herein, the term “free base” refers to the neutral conjugate base of an organic acid. Non-limiting examples include a neutral amine (R3N), guanidine (R2N(C═NR)NR2), and an imine (R(C═NR)R), inter alia, wherein R may be H or an organic group.

The phrase “inhibit” as used herein, means to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

The term “long COVID” also known as “Post-Acute Sequelae of SARS-CoV-2 infection (PASC),” “chronic COVID syndrome (CCS),” and “long-haul COVID,” as used herein, refers to a condition whereby an individual apparently affected by a COVID-19 infection (i.e., displaying at least one symptom of a COVID-19 infection) does not recover for an extended period of time (e.g., several weeks or months after the typical convalescence period of COVID-19) following the onset of one or more symptoms suggestive of COVID-19, regardless of whether or not the individual has been tested for SARS-CoV-2. Persistent symptoms include, but are not limited to, fatigue, headaches, shortness of breath, anosmia (loss of smell), muscle weakness, low fever, and cognitive dysfunction (i.e., brain fog). Studies suggest that approximately 10% of people who tested positive for SARS-CoV-2 experienced one or more symptoms for longer than 12 weeks. Anyone infected with SARS-CoV-2 can suffer from long COVID after the infection is considered to have ended, including young, healthy people, and even if the initial disease was mild.

As used herein, the term “metabolite” refers to an intermediate or end product of a metabolic process, including, but not limited to, hydrolysis, esterification, conjugation, oxidation, and reduction.

As used herein, the term “pharmaceutically acceptable” refers to a material, such as a carrier or diluent, which does not abrogate the biological activity or properties of the compound useful within the disclosure, and is relatively non-toxic, i.e., the material may be administered to a subject without causing undesirable biological effects or interacting in a deleterious manner with any one of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the disclosure, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the disclosure, and are physiologically acceptable to the subject. Supplementary active compounds may also be incorporated into the compositions. The “pharmaceutically acceptable carrier” may further include a pharmaceutically acceptable salt of the compound useful within the disclosure. Other additional ingredients that may be included in the pharmaceutical compositions used in the practice of the disclosure are known in the art and described, for example in Remington's Pharmaceutical Sciences (Genaro, Ed., Mack Publishing Co., 1985, Easton, Pa.), which is incorporated herein by reference.

As used herein, the language “pharmaceutically acceptable salt” refers to a salt of the administered compound prepared from pharmaceutically acceptable non-toxic acids and/or bases, including inorganic acids, inorganic bases, organic acids, inorganic bases, solvates (including hydrates) and clathrates thereof.

The terms “pharmaceutically effective amount” and “effective amount” refer to a non-toxic but sufficient amount of an agent to provide the desired biological result. That result can be reduction and/or alleviation of the signs, symptoms, or causes of a disease or disorder, or any other desired alteration of a biological system.

The term “recovery time” as used herein refers to an amount of time between the onset of a disease and/or disorder, including but not limited to an infection, and a return to a healthy or convalescent state with respect to said disease and/or disorder.

As used herein, the terms “RT-PCR” or “reverse transcription polymerase chain reaction” refer to a laboratory technique combining reverse transcription of the RNA present in a sample to DNA, with amplification of specific DNA targets using the polymerase chain reaction. These terms may also refer to real time PCR, wherein the amplification of the DNA target is monitored and quantified by at least one of several detection methods, such methods comprising non-specific fluorescent dye intercalation with DNA and sequence-specific DNA probes consisting of oligonucleotides labeled with a fluorescent reporter, wherein fluorescence is detected only upon hybridization of the probe with its complementary sequence.

By the term “specifically binds” as used herein, is meant a molecule, such as an antibody, which recognizes and binds to another molecule or feature, but does not substantially recognize or bind other molecules or features in a sample.

The terms “subject” or “patient” or “individual” for the purposes of the present disclosure includes humans and other animals, particularly mammals, and other organisms. Thus the methods are applicable to both human therapy and veterinary applications.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The terms “treat,” “treating,” and “treatment,” refer to one or more therapeutic or palliative measures described herein. The methods of “treatment” employ administration to a subject, in need of such treatment, a composition, for example, a subject afflicted with a disease or disorder, or a subject who has one or symptoms of such a disease or disorder, in order to cure, delay, reduce the severity of, or ameliorate one or more symptoms of the disorder or recurring disorder, or in order to prolong the survival of a subject beyond that expected in the absence of such treatment.

As used herein, the term “trough,” as related to a drug that is administered periodically to a subject, corresponds to the circulating drug concentration in the subject just before the administration of the next dose of drug to the subject.

As used herein, the term “viral load” refers to an amount or concentration of a virus in a sample of blood, saliva, mucus, or other bodily fluid, and is often expressed as a ratio of viral particles per volume of bodily fluid.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, and 6.

### DESCRIPTION

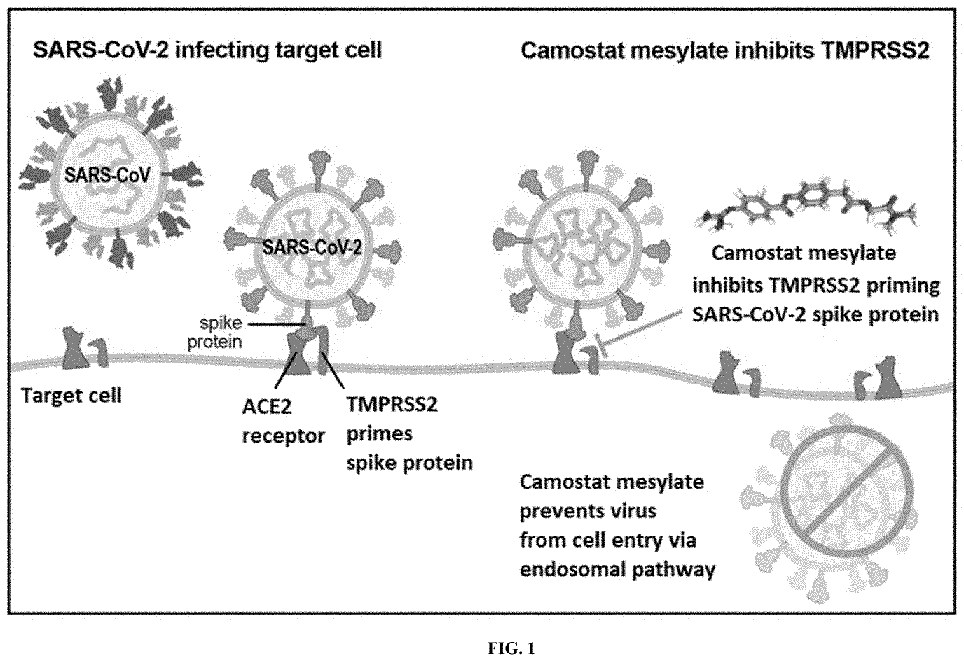

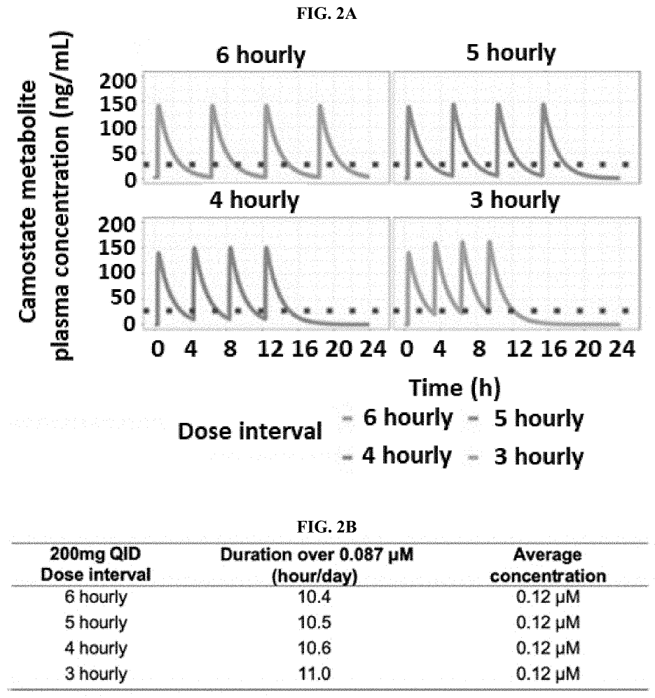

SARS-CoV-2 uses the angiotensin converting enzyme II (ACE2) as its cell entry receptor protein to access and infect human cells. The interaction between ACE2 and the spike protein is not in ACE2's active site. This process critically uses the human epithelial cell (respiratory, gastrointestinal tract) surface-expressed transmembrane serine protease 2 (TMPRSS2). Utilizing research on severe acute respiratory syndrome coronavirus (SARS-CoV) and the closely related SARS-CoV-2 cell entry mechanism, it was demonstrated that SARS-CoV-2 cellular entry can be blocked in vitro as well as in vivo by the serine protease inhibitor camostat mesylate (FIG. 1).

Camostat mesylate is used primarily in the treatment of postoperative reflux esophagitis and for acute, symptomatic exacerbations of chronic pancreatitis. In Japan, camostat mesylate is approved for acute symptomatic exacerbation of chronic pancreatitis at a dose of 600 mg po/day, and for postoperative reflux esophagitis at 300 mg po/day. Camostat mesylate is well tolerated and has no known drug-drug interactions. Reported adverse effect are rare (<3%) and typically mild, such as pruritus, increased thirst and appetite, and lightheadedness. Using a strain of SARS-CoV-1 adapted to cause lethal disease in a mouse model, camostat mesylate delivered at a concentration similar to the clinically achievable concentration in humans reduced mortality from 100% to 30-35% in mice following SARS-CoV-1 infection.

A recent publication demonstrated that use of camostat mesylate tested in hospitalized COVID-19 patients in a double-blind randomized placebo-controlled trial with camostast mesylate given 48 hours of admission (200 mg po three times daily for 5 days) did not have an observable effect on clinical severity, for example, on escalation to ICU admission. Nevertheless, adverse effects were not observed with the use of camostat mesylate (EClinicalMedicine, 2021, 35, 100849; NCT04321096).

Additionally, a multi-center, randomized, double-blind, placebo-controlled, parallel group Phase III study in patients with asymptomatic to moderate COVID-19 was conducted, wherein patients received camostat mesylate (600 mg po four times daily for up to 14 days). The primary endpoint of the study was time required for a negative SARS-CoV-2 test, and camostat mesylate did not show efficacy for that primary endpoint (www dot ono-pharma dot com/news/20210611 dot html).

The goal of the randomized, double-blind, placebo controlled phase II clinical trial described herein was to determine whether camostat mesylate reduces SARS-COV-2 viral load in early COVID-19 disease. Furthermore, pre-specified secondary analyses were to determine whether there might be beneficial clinical effects of camostat mesylate on COVID-19 symptom scores, even though it had initially been speculated that the number of subjects in this study may be insufficient to detect small effects.

In the randomized, double-blind, placebo-controlled phase II trial described herein, it has been demonstrated that, using a high-resolution scoring system (i.e., COVID-19 PRO self-score), camostat mesylate accelerated COVID-19 symptom resolution by up to 5 days. This patient-focused outcome supports expanded clinical trial testing of camostat mesylate for the early treatment of COVID-19, especially to determine whether this drug reduces risk for severe disease and hospitalization. The potential importance of oral treatment of early COVID-19 with an inexpensive, repurposed drug with minimal side effects and no drug-drug interaction, to reduce risk of complications, cannot be overstated in the context of the ongoing pandemic.

The present disclosure relates to the discovery that administration of camostat mesylate is useful for treating, ameliorating, shortening the duration, and/or reversing at least one symptom and/or complication in a human subject with a coronavirus infection, and/or preventing and/or reducing the occurrence of long COVID in a human subject with a SARS-CoV-2 infection.

Compositions

In certain embodiments, the present disclosure provides a composition comprising camostat. Camostat refers to 4-[[4-[(Aminoiminomethyl)amino]benzoyl]oxy]benzeneacetic acid 2-(dimethylamino)-2-oxoethyl ester, whereas camostat mesylate refers to the mesylate (i.e., methanesulfonate) salt of camostat.

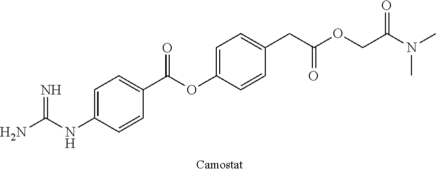 [SMILES: CN(C)C(=O)COC(=O)Cc1ccc(OC(=O)c2ccc(NC(=N)N)cc2)cc1]

In certain embodiments, the disclosure provides a pharmaceutical composition comprising about 645 mg of camostat free base, or an equimolar amount of a pharmaceutically acceptable salt or solvate thereof. In certain embodiments, the disclosure provides a pharmaceutical composition comprising about 800 mg of camostat mesylate.

In certain embodiments, the composition comprises a single administration dose comprising about 645 mg of camostat free base, or an equimolar amount of a pharmaceutically acceptable salt or solvate thereof. In certain embodiments, the composition comprises a single administration dose comprising about 800 mg of camostat mesylate.

In certain embodiments, the composition comprises two identical administration doses totaling about 645 mg of camostat free base, or an equimolar amount of a pharmaceutically acceptable salt or solvate thereof. In certain embodiments, the composition comprises two identical administration doses comprising about 800 mg of camostat mesylate.

In certain embodiments, the composition comprises three identical administration doses totaling about 645 mg of camostat free base, or an equimolar amount of a pharmaceutically acceptable salt or solvate thereof. In certain embodiments, the composition comprises three identical administration doses comprising about 800 mg of camostat mesylate.

In certain embodiments, the composition comprises four identical administration doses totaling about 645 mg of camostat free base, or an equimolar amount of a pharmaceutically acceptable salt or solvate thereof. In certain embodiments, the composition comprises four identical administration doses comprising about 800 mg of camostat mesylate.

In certain embodiments, the composition comprises five or more identical administration doses totaling about 645 mg of camostat free base, or an equimolar amount of a pharmaceutically acceptable salt or solvate thereof. In certain embodiments, the composition comprises five or more identical administration doses comprising about 800 mg of camostat mesylate.

Camostat is a proteolytic enzyme inhibitor that is used primarily in the treatment of postoperative reflux esophagitis and for acute, symptomatic exacerbations of chronic pancreatitis.

Camostat (and its salts) is immediately and extensively hydrolyzed in plasma to 4-(4-guanidinobenzoyl-oxy)phenylacetic acid (GBPA), which is further degraded to 4-guanidinobenzoic acid (GBA). Camostat and GBPA have similar biological activity, while GBA is inactive. Excretion is mainly renal as GBA (>95%).

Camostat is a serine protease inhibitor, which inhibits the protease transmembrane protease serine S1 member 2 (TMPRSS2). In humans, TMPRSS2 activates the spike protein of the severe acute respiratory syndrome coronavirus (SARS-CoV) on the cell surface, and camostat inhibits TMPRSS2-dependent infection by SARS-CoV. Unfortunately, there is no clinical experience in using camostat (or its mesylate salt) in humans to treat SARS-CoV-2.

Compounds described herein can also include isotopically labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ¹³N, ¹⁵N, ¹⁵O, ¹⁷O, and ¹⁸O. In certain embodiments, substitution with heavier isotopes such as deuterium affords greater chemical stability. Isotopically labeled compounds are prepared by any suitable method or by processes using an appropriate isotopically labeled reagent in place of the non-labeled reagent otherwise employed.

In certain embodiments, the compounds described herein are labeled by other means, including, but not limited to, the use of chromophores or fluorescent moieties, bioluminescent labels, and/or chemiluminescent labels.

Salts

The compounds described herein may form salts with acids, and such salts are included in the present disclosure. The term “salts” embraces addition salts of free acids that are useful within the methods of the disclosure. The term “pharmaceutically acceptable salt” refers to salts that possess toxicity profiles within a range that affords utility in pharmaceutical applications. In certain embodiments, the salts are pharmaceutically acceptable salts. Pharmaceutically unacceptable salts may nonetheless possess properties such as high crystallinity, which have utility in the practice of the present disclosure, such as for example utility in process of synthesis, purification or formulation of compounds useful within the methods of the disclosure.

Suitable pharmaceutically acceptable acid addition salts may be prepared from an inorganic acid or from an organic acid. Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (or pamoic), methanesulfonic, ethanesulfonic, benzenesulfonic, pantothenic, sulfanilic, 2-hydroxyethanesulfonic, trifluoromethanesulfonic, p-toluenesulfonic, cyclohexylaminosulfonic, stearic, alginic, β-hydroxybutyric, salicylic, galactaric, galacturonic acid, glycerophosphonic acids and saccharin (e.g., saccharinate, saccharate). Salts may be comprised of a fraction of one, one or more than one molar equivalent of acid or base with respect to any compound of the disclosure.

Methods

The present disclosure relates in one aspect to a method of treating, ameliorating, shortening duration, and/or reversing at least one symptom and/or complication in a human subject with a coronavirus infection.

The present disclosure relates in one aspect to a method of preventing and/or reducing the occurrence of long COVID, and/or a symptom and/or complication thereof, hospitalization, and/or death in a human subject with a SARS-CoV-2 infection.

In certain embodiments, the coronavirus is at least one of MERS-CoV, SARS-CoV, and/or SARS-CoV-2. In certain embodiments, the coronavirus is SARS-CoV-2.

In certain embodiments, the method comprises administering to the human a dose of about 645 mg of camostat free base, or an equimolar amount of a pharmaceutically acceptable salt or solvate thereof (“drug” hereinafter). In certain embodiments, the dose of the drug comprises about 800 mg of camostat mesylate.

In certain embodiments, the subject suffers from long COVID.

In certain embodiments, the administering reverses, reduces and/or prevents progression of the coronavirus infection. In certain embodiments, progression of the coronavirus infection comprises hospitalization and/or death.

In certain embodiments, the administering reduces and/or eliminates at least one symptom of the coronavirus infection.

In certain embodiments, the administering reduces recovery time for at least one symptom of the coronavirus infection. In certain embodiments, the reduction in recovery time relates to a comparison between a subjected treated according to the methods of the present disclosure and a control subject. In certain embodiments, the control subject has not been treated. In certain embodiments, the control subject has been treated with a placebo.

In certain embodiments, the at least one symptom is selected from the group consisting of runny nose, congested nose, sinus pressure, sneezing, scratchy or itchy throat, sore or painful throat, swollen throat, difficulty swallowing, teary or water eyes, sore or painful eyes, eyes sensitive to light, difficulty breathing, chest congestion, chest tightness, dry or hacking cough, wet or loose cough, frequent coughing, coughing mucus or phlegm, lack of appetite, gastrointestinal discomfort (i.e., stomach ache), vomiting, diarrhea, headache, head congestion, dizziness, lightheadedness, nausea, excessive sleeping, dyspnea, myalgia, fever, difficulty sleeping, body aches or pains, fatigue, chills or shivering, feeling cold, feeling hot, sweating, discomfort, abnormal, reduced, or eliminated sense of smell (e.g., anosmia), and abnormal, reduced, or eliminated sense of taste (e.g., ageusia).

In certain embodiments, the at least one symptom comprises anosmia. In certain embodiments, the at least one symptom comprises ageusia.

In certain embodiments, the recovery time is reduced by at least about 1 day. In certain embodiments, the recovery time is reduced by at least about 2 days. In certain embodiments, the recovery time is reduced by at least about 3 days. In certain embodiments, the recovery time is reduced by at least about 4 days. In certain embodiments, the recovery time is reduced by at least about 5 days. In certain embodiments, the recovery time is reduced by at least about 6 days. In certain embodiments, the recovery time is reduced by at least about 7 days. In certain embodiments, the recovery time is reduced by at least about 8 days. In certain embodiments, the recovery time is reduced by at least about 9 days. In certain embodiments, the recovery time is reduced by at least about 10 days. In certain embodiments, the recovery time is reduced as compared to a control human subject who is not administered the daily dose of about 645 mg of camostat free base, or an equimolar amount of a pharmaceutically acceptable salt or solvate thereof.

In certain embodiments, the administration to the subject is by at least one route selected from the group consisting of nasal, inhalational, topical, oral, buccal, rectal, pleural, peritoneal, vaginal, intramuscular, subcutaneous, transdermal, epidural, intratracheal, otic, intraocular, intrathecal, and intravenous routes. In other embodiments, the administration to the subject is by an oral route.

In certain embodiments, the administration to the subject is performed daily. In certain embodiments, the dose contemplated in the disclosure is a daily dose.

In certain embodiments, the administration to the subject is performed for a period of 7 days.

In certain embodiments, the daily administration comprises about 800 mg of camostat mesylate. In certain embodiments, the daily administration comprises four approximately equal doses of the drug (e.g., camostat mesylate). For example, the four equal doses of camostat free base can be about 161.25 mg each. In other embodiments, each of the four equal doses of camostat mesylate is about 200 mg of camostat mesylate.

In certain embodiments, each dose of the drug is administered within 3 hours of a previous and/or following dose. For example, a first dose of the drug is administered to the subject and a second dose of the drug is administered to the subject within three hours of the first dose. Each of the third and fourth doses of the drug are independently administered to the subject within three hours of the preceding dose. In other embodiments, each dose of the drug is administered within 4 hours of a previous and/or following dose. In yet other embodiments, each dose of the drug is administered within 5 hours of a previous and/or following dose. In yet other embodiments, each dose of the drug is administered within 6 hours of a previous and/or following dose.

In certain embodiments, the administration of the drug affords a trough plasma concentration of GBPA in the subject selected from the group consisting of about 0.1 ng/ml to about 5 ng/ml, about 5 ng/ml to about 10 ng/ml, about 10 ng/ml to about 15 ng/ml, about 15 ng/ml to about 20 ng/ml, about 20 ng/ml to about 25 ng/ml, and about 5 ng/ml to about 25 ng/ml. In certain embodiments, the administration of the drug to the subject affords an average plasma concentration of GBPA in the subject is about 0.09 μM to about 0.13 μM. In certain embodiments, the plasma concentration of GBPA in the subject is selected from the group consisting of about 0.09 μM to about 0.10 μM, 0.10 μM to about 0.11 about 0.11 μM to about 0.12 about 0.12 μM to about 0.13 and about 0.11 μM to about 0.13 μM.

In certain embodiments, the administration of the drug to the subject affords a maximal plasma concentration of GBPA in the subject is about 140 ng/ml to about 160 ng/ml. In certain embodiments, the maximal plasma concentration of GBPA in the subject is selected from the group consisting of about 140 ng/ml to about 150 ng/ml and about 150 ng/ml to about 160 ng/ml.

Pharmaceutical Compositions and Formulations

The disclosure provides pharmaceutical compositions comprising camostat or a salt or solvate thereof, which are useful to practice methods of the disclosure. Such a pharmaceutical composition may comprise camostat or a salt or solvate thereof, in a form suitable for administration to a subject, or the pharmaceutical composition may comprise camostat or a salt or solvate thereof, and one or more pharmaceutically acceptable carriers, one or more additional ingredients, or any combinations of these. Camostat may be present in the pharmaceutical composition in the form of a physiologically acceptable salt, such as in combination with a physiologically acceptable cation or anion, as is well known in the art.

In certain embodiments, the pharmaceutical compositions useful for practicing the method of the disclosure may be administered to deliver a dose of between 1 ng/kg/day and 100 mg/kg/day. In other embodiments, the pharmaceutical compositions useful for practicing the disclosure may be administered to deliver a dose of between 1 ng/kg/day and 1,000 mg/kg/day.

The relative amounts of the active ingredient, the pharmaceutically acceptable carrier, and any additional ingredients in a pharmaceutical composition of the disclosure will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the disclosure may be suitably developed for nasal, inhalational, oral, rectal, vaginal, pleural, peritoneal, parenteral, topical, transdermal, pulmonary, intranasal, buccal, ophthalmic, epidural, intrathecal, intravenous, or another route of administration. A composition useful within the methods of the disclosure may be directly administered to the brain, the brainstem, or any other part of the central nervous system of a mammal or bird. Other contemplated formulations include projected nanoparticles, microspheres, liposomal preparations, coated particles, polymer conjugates, resealed erythrocytes containing the active ingredient, and immunologically-based formulations.

In certain embodiments, the compositions of the disclosure are part of a pharmaceutical matrix, which allows for manipulation of insoluble materials and improvement of the bioavailability thereof, development of controlled or sustained release products, and generation of homogeneous compositions. By way of example, a pharmaceutical matrix may be prepared using hot melt extrusion, solid solutions, solid dispersions, size reduction technologies, molecular complexes (e.g., cyclodextrins, and others), microparticulate, and particle and formulation coating processes. Amorphous or crystalline phases may be used in such processes.

The route(s) of administration will be readily apparent to the skilled artisan and will depend upon any number of factors including the type and severity of the disease being treated, the type and age of the veterinary or human patient being treated, and the like.

The formulations of the pharmaceutical compositions described herein may be prepared by any method known or hereafter developed in the art of pharmacology and pharmaceutics. In general, such preparatory methods include the step of bringing the active ingredient into association with a carrier or one or more other accessory ingredients, and then, if necessary or desirable, shaping or packaging the product into a desired single-dose or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the disclosure is contemplated include, but are not limited to, humans and other primates, mammals including commercially relevant mammals such as cattle, pigs, horses, sheep, cats, and dogs.

In certain embodiments, the compositions of the disclosure are formulated using one or more pharmaceutically acceptable excipients or carriers. In certain embodiments, the pharmaceutical compositions of the disclosure comprise a therapeutically effective amount of at least one compound of the disclosure and a pharmaceutically acceptable carrier. Pharmaceutically acceptable carriers, which are useful, include, but are not limited to, glycerol, water, saline, ethanol, recombinant human albumin (e.g., RECOMBUMIN®), solubilized gelatins (e.g., GELOFUSINE®), and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), recombinant human albumin, solubilized gelatins, suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, isotonic agents, for example, sugars, sodium chloride, or polyalcohols such as mannitol and sorbitol, are included in the composition. Prolonged absorption of the injectable compositions may be brought about by including in the composition an agent that delays absorption, for example, aluminum monostearate or gelatin.

Formulations may be employed in admixtures with conventional excipients, i.e., pharmaceutically acceptable organic or inorganic carrier substances suitable for oral, parenteral, nasal, inhalational, intravenous, subcutaneous, transdermal enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring, and/or fragrance-conferring substances and the like. They may also be combined where desired with other active agents, e.g., other analgesic, anxiolytics or hypnotic agents. As used herein, “additional ingredients” include, but are not limited to, one or more ingredients that may be used as a pharmaceutical carrier.

The composition of the disclosure may comprise a preservative from about 0.005% to 2.0% by total weight of the composition. The preservative is used to prevent spoilage in the case of exposure to contaminants in the environment. Examples of preservatives useful in accordance with the disclosure include but are not limited to those selected from the group consisting of benzyl alcohol, sorbic acid, parabens, imidurea and any combinations thereof. One such preservative is a combination of about 0.5% to 2.0% benzyl alcohol and 0.05-0.5% sorbic acid.

The composition may include an antioxidant and a chelating agent that inhibit the degradation of the compound. Antioxidants for some compounds are BHT, BHA, alpha-tocopherol and ascorbic acid in the exemplary range of about 0.01% to 0.3%, or BHT in the range of 0.03% to 0.1% by weight by total weight of the composition. The chelating agent may be present in an amount of from 0.01% to 0.5% by weight by total weight of the composition. Exemplary chelating agents include edetate salts (e.g. disodium edetate) and citric acid in the weight range of about 0.01% to 0.20%, or in the range of 0.02% to 0.10% by weight by total weight of the composition. The chelating agent is useful for chelating metal ions in the composition that may be detrimental to the shelf life of the formulation. While BHT and disodium edetate are exemplary antioxidant and chelating agent, respectively, for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethyl cellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, acacia, and ionic or non-ionic surfactants. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the disclosure may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the disclosure may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, ionic and non-ionic surfactants, and a preservative. Additional excipients, such as fillers and sweetening, flavoring, or coloring agents, may also be included in these formulations.

A pharmaceutical composition of the disclosure may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying. Methods for mixing components include physical milling, the use of pellets in solid and suspension formulations and mixing in a transdermal patch, as known to those skilled in the art.

Administration/Dosing

The regimen of administration may affect what constitutes an effective amount. The therapeutic formulations may be administered to the patient either prior to or after the onset of a disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation. Administration of the compositions of the present disclosure to a patient, such as a mammal, such as a human, may be carried out using known procedures, at dosages and for periods of time effective to treat, ameliorate, and/or prevent a disease or disorder contemplated herein. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the disclosure is from about 0.01 mg/kg to 100 mg/kg of body weight/per day.

The compound may be administered to an animal as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon a number of factors, such as, but not limited to, type and severity of the disease being treated, and type and age of the animal.

Actual dosage levels of the active ingredients in the pharmaceutical compositions of this disclosure may be varied so as to obtain an amount of the active ingredient that is effective to achieve the desired therapeutic response for a particular patient, composition, and mode of administration, without being toxic to the patient.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the disclosure are dictated by and directly dependent on (a) the unique characteristics of the therapeutic compound and the particular therapeutic effect to be achieved, and (b) the limitations inherent in the art of compounding/formulating such a therapeutic compound for the treatment of a disease or disorder in a patient.

Compounds of the disclosure for administration may be in the range of from about 1 μg to about 7,500 mg, about 20 μg to about 7,000 mg, about 40 μg to about 6,500 mg, about 80 μg to about 6,000 mg, about 100 μg to about 5,500 mg, about 200 μg to about 5,000 mg, about 400 g to about 4,000 mg, about 800 μg to about 3,000 mg, about 1 mg to about 2,500 mg, about 2 mg to about 2,000 mg, about 5 mg to about 1,000 mg, about 10 mg to about 750 mg, about 20 mg to about 600 mg, about 30 mg to about 500 mg, about 40 mg to about 400 mg, about 50 mg to about 300 mg, about 60 mg to about 250 mg, about 70 mg to about 200 mg, about 80 mg to about 150 mg, and any and all whole or partial increments there-in-between.

In some embodiments, the dose of a compound of the disclosure is from about 0.5 μg and about 5,000 mg. In some embodiments, a dose of a compound of the disclosure used in compositions described herein is less than about 5,000 mg, or less than about 4,000 mg, or less than about 3,000 mg, or less than about 2,000 mg, or less than about 1,000 mg, or less than about 800 mg, or less than about 600 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present disclosure is directed to a packaged pharmaceutical composition comprising a container holding a therapeutically effective amount of a compound of the disclosure, alone or in combination with a second pharmaceutical agent; and instructions for using the compound to treat, prevent, or reduce one or more symptoms of a disease or disorder in a patient.

The term “container” includes any receptacle for holding the pharmaceutical composition or for managing stability or water uptake. For example, in certain embodiments, the container is the packaging that contains the pharmaceutical composition, such as liquid (solution and suspension), semisolid, lyophilized solid, solution and powder or lyophilized formulation present in dual chambers. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing a disease or disorder in a patient.

Administration

Routes of administration of any one of the compositions of the disclosure include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, epidural, intrapleural, intraperitoneal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, emulsions, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

Oral Administration

For oral application, particularly suitable are tablets, dragees, liquids, drops, capsules, caplets and gelcaps. Other formulations suitable for oral administration include, but are not limited to, a powdered or granular formulation, an aqueous or oily suspension, an aqueous or oily solution, a paste, a gel, toothpaste, a mouthwash, a coating, an oral rinse, or an emulsion. The compositions intended for oral use may be prepared according to any method known in the art and such compositions may contain one or more agents selected from the group consisting of inert, non-toxic, generally recognized as safe (GRAS) pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically controlled release tablets. Tablets may further comprise a sweetening agent, a flavoring agent, a coloring agent, a preservative, or some combination of these in order to provide for pharmaceutically elegant and palatable preparation. Hard capsules comprising the active ingredient may be made using a physiologically degradable composition, such as gelatin. The capsules comprise the active ingredient, and may further comprise additional ingredients including, for example, an inert solid diluent such as calcium carbonate, calcium phosphate, or kaolin.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin from animal-derived collagen or from a hypromellose, a modified form of cellulose, and manufactured using optional mixtures of gelatin, water and plasticizers such as sorbitol or glycerol. Such soft capsules comprise the active ingredient, which may be mixed with water or an oil medium such as peanut oil, liquid paraffin, or olive oil.

For oral administration, the compounds of the disclosure may be in the form of tablets or capsules prepared by conventional means with pharmaceutically acceptable excipients such as binding agents; fillers; lubricants; disintegrates; or wetting agents. If desired, the tablets may be coated using suitable methods and coating materials such as OPADRY® film coating systems available from Colorcon, West Point, Pa. (e.g., OPADRY® OY Type, OYC Type, Organic Enteric OY-P Type, Aqueous Enteric OY-A Type, OY-PM Type and OPADRY® White, 32K18400). It is understood that similar type of film coating or polymeric products from other companies may be used.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface-active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycolate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e., having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e., drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

The present disclosure also includes a multi-layer tablet comprising a layer providing for the delayed release of one or more compounds useful within the methods of the disclosure, and a further layer providing for the immediate release of one or more compounds useful within the methods of the disclosure. Using a wax/pH-sensitive polymer mix, a gastric insoluble composition may be obtained in which the active ingredient is entrapped, ensuring its delayed release.

Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl para-hydroxy benzoates or sorbic acid). Liquid formulations of a pharmaceutical composition of the disclosure which are suitable for oral administration may be prepared, packaged, and sold either in liquid form or in the form of a dry product intended for reconstitution with water or another suitable vehicle prior to use.

Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Injectable formulations may also be prepared, packaged, or sold in devices such as patient-controlled analgesia (PCA) devices. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3-butanediol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form in a recombinant human albumin, a fluidized gelatin, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

Topical Administration

An obstacle for topical administration of pharmaceuticals is the stratum corneum layer of the epidermis. The stratum corneum is a highly resistant layer comprised of protein, cholesterol, sphingolipids, free fatty acids and various other lipids, and includes cornified and living cells. One of the factors that limit the penetration rate (flux) of a compound through the stratum corneum is the amount of the active substance that can be loaded or applied onto the skin surface. The greater the amount of active substance which is applied per unit of area of the skin, the greater the concentration gradient between the skin surface and the lower layers of the skin, and in turn the greater the diffusion force of the active substance through the skin. Therefore, a formulation containing a greater concentration of the active substance is more likely to result in penetration of the active substance through the skin, and more of it, and at a more consistent rate, than a formulation having a lesser concentration, all other things being equal.

Formulations suitable for topical administration include, but are not limited to, liquid or semi-liquid preparations such as liniments, lotions, oil-in-water or water-in-oil emulsions such as creams, ointments or pastes, and solutions or suspensions. Topically administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

Enhancers of permeation may be used. These materials increase the rate of penetration of drugs across the skin. Typical enhancers in the art include ethanol, glycerol monolaurate, PGML (polyethylene glycol monolaurate), dimethylsulfoxide, and the like. Other enhancers include oleic acid, oleyl alcohol, ethoxydiglycol, laurocapram, alkanecarboxylic acids, dimethylsulfoxide, polar lipids, or N-methyl-2-pyrrolidone.

One acceptable vehicle for topical delivery of some of the compositions of the disclosure may contain liposomes. The composition of the liposomes and their use are known in the art (i.e., U.S. Pat. No. 6,323,219).

In alternative embodiments, the topically active pharmaceutical composition may be optionally combined with other ingredients such as adjuvants, anti-oxidants, chelating agents, surfactants, foaming agents, wetting agents, emulsifying agents, viscosifiers, buffering agents, preservatives, and the like. In other embodiments, a permeation or penetration enhancer is included in the composition and is effective in improving the percutaneous penetration of the active ingredient into and through the stratum corneum with respect to a composition lacking the permeation enhancer. Various permeation enhancers, including oleic acid, oleyl alcohol, ethoxydiglycol, laurocapram, alkanecarboxylic acids, dimethylsulfoxide, polar lipids, or N-methyl-2-pyrrolidone, are known to those of skill in the art. In another aspect, the composition may further comprise a hydrotropic agent, which functions to increase disorder in the structure of the stratum corneum, and thus allows increased transport across the stratum corneum. Various hydrotropic agents such as isopropyl alcohol, propylene glycol, or sodium xylene sulfonate, are known to those of skill in the art.

The topically active pharmaceutical composition should be applied in an amount effective to affect desired changes. As used herein “amount effective” shall mean an amount sufficient to cover the region of skin surface where a change is desired. An active compound should be present in the amount of from about 0.0001% to about 15% by weight volume of the composition. For example, it should be present in an amount from about 0.0005% to about 5% of the composition; for example, it should be present in an amount of from about 0.001% to about 1% of the composition. Such compounds may be synthetically- or naturally derived.

Buccal Administration

A pharmaceutical composition of the disclosure may be prepared, packaged, or sold in a formulation suitable for buccal administration. Such formulations may, for example, be in the form of tablets or lozenges made using conventional methods, and may contain, for example, 0.1 to 20% (w/w) of the active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, may have an average particle or droplet size in the range from about 0.1 to about 200 nanometers, and may further comprise one or more of the additional ingredients described herein. The examples of formulations described herein are not exhaustive and it is understood that the disclosure includes additional modifications of these and other formulations not described herein, but which are known to those of skill in the art.

Rectal Administration

A pharmaceutical composition of the disclosure may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants, and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants, and preservatives.

Additional Administration Forms

Additional dosage forms of this disclosure include dosage forms as described in U.S. Pat. Nos. 6,340,475, 6,488,962, 6,451,808, 5,972,389, 5,582,837, and 5,007,790. Additional dosage forms of this disclosure also include dosage forms as described in U.S. Patent Applications Nos. 20030147952, 20030104062, 20030104053, 20030044466, 20030039688, and 20020051820. Additional dosage forms of this disclosure also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

Controlled Release Formulations and Drug Delivery Systems

In certain embodiments, the compositions and/or formulations of the present disclosure may be, but are not limited to, short-term, rapid-onset and/or rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the disclosure may be administered in the form of microparticles, for example, by injection or in the form of wafers or discs by implantation.

In certain embodiments of the disclosure, the compounds useful within the disclosure are administered to a subject, alone or in combination with another pharmaceutical agent, using a sustained release formulation.

The term delayed release is used herein in its conventional sense to refer to a drug formulation that provides for an initial release of the drug after some delay following drug administration and that may, although not necessarily, include a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Those skilled in the art will recognize or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

### EXPERIMENTAL EXAMPLES

Various embodiments of the present application can be better understood by reference to the following Examples which are offered by way of illustration. The scope of the present application is not limited to the Examples given herein.

Materials and Methods

Phase IIa Double-Blind Placebo-Controlled Study of Camostat Mesylate

The phase IIa double-blind placebo-controlled study of camostat mesylate was performed with 70 subjects with RT-PCR-confirmed COVID-19 infection. Subjects were enrolled in two cohorts. The treatment cohort consists of subjects receiving camostat mesylate, and the control cohort consists of subjects receiving a placebo. The treatment cohort received 200 mg camostat mesylate four times daily for seven days. The control cohort received a placebo four times daily for seven days.

The major inclusion criteria were age 18 or older, a first positive COVID-19 RT-PCR assay within the previous 72 hours, associated with at least one COVID-19-compatible symptom such as fever, upper respiratory symptoms, cough, chills, loss of taste/smell, or a recent high-risk exposure to COVID-19. Major exclusion criteria were hospitalized patients with COVID-19 and pregnancy or lactation.

The Phase IIa clinical trial collected nasopharyngeal swabs and saliva from all subjects of both cohorts on study days 0, 2, 4, 6, 14 (±2), and 28 (±2). Nasopharyngeal swab and saliva samples were analyzed by an approved COVID-19 RT-PCR assay (primary end-point). Quidel's Sofia platform is used at the study site for antigen detection. Positivity and Ct values are recorded and log₁₀ viral loads back-extrapolated from log₁₀ being equivalent to 3.3 Ct units. Additionally, all subjects self-administered Likert-type symptom score evaluations daily from days 0-14 (secondary end-point).

The Phase IIa clinical trial collected a total of 125 mL of blood from all subjects of both cohorts on each of days 0, 14(±2), and 28(±2). Clinical surveillance of blood includes complete blood count and blood chemistry analysis, including electrolytes, BUN, creatinine, AST, ALT, total and direct bilirubin, and alkaline phosphatase.

Camostat Mesylate

Camostat mesylate is provided as 500 tablets/bottle. Each tablet contained 100 mg of Camostat mesylate. For study administration, two tablets were combined into one 200 mg capsule. For blinding, two intact active 100 mg camostat mesylate tablets are placed in a capsule shell, back filled with a sufficient quantity of microcrystalline cellulose, and closed. A matching placebo was compounded, using a matching empty capsule filled with a sufficient quantity of microcrystalline cellulose. All capsules were visually inspected and packed into polypropylene bottles.

Sample Size

The sample size calculation was based on the primary outcome of interest; specifically, a change in the log₁₀ respiratory (nasopharyngeal swab and saliva swab RT-PCR) viral load from baseline to day 4 post-randomization. Given the limited data on the variability of the change in log 10 viral load, the study was based on detecting a moderate standardized effect size of 0.3 using an analysis of covariance (ANCOVA) and adjusting for baseline log₁₀ viral load. To elaborate, one scenario that would produce a 0.3 standardized effect size would be a change of 4 in the log₁₀ viral load in the camostat mesylate group compared to a change of 1 in the log₁₀ viral load in the placebo group assuming a standard deviation of 5.0. For ANCOVA, the effect size was the standard deviation of the treatment means divided by the pooled standard deviations of the observations. To be conservative, a R-squared of 0 between the log₁₀ viral RNA at 4 days and baseline log₁₀ viral RNA was assumed. With a power of 90%, and a type I error rate of 10% (2-sided), detection of the hypothesized 0.3 standardized effect size was reasoned to occur with 98 patients divided into 49 patients per group with a 1:1 randomization. Increasing this sample size by 15%, wherein 5% is provided for an efficacy and futility look at 50% information (i.e., when half of the patients have been enrolled) and 10% is provided to account for loss to follow up, gives a total of 114 participants (57 per treatment arm). The present study was ended with 70 participants.

Assessment of Adverse Events

Adverse events of the present study were assessed by verbal report, and specifically sought according to the approval package insert. All adverse events must have their relationship to the study intervention assessed by the clinician who examines and evaluates the participant based on the temporal relationship and his/her clinical judgement. The degree of certainty about causality is graded related or not related.

Symptom Score Evaluation

Subjects self-administered Likert-type symptom score evaluations daily from days 0-14, wherein subjects provided a ranking (i.e., 0—not at all, 1—a little bit, 2—somewhat, 3—quite a bit, and 4—very much) in response to various symptoms (e.g., nose, throat, eyes, chest/respiratory, gastrointestinal, body/systemic, and smell/taste symptoms) (Table 1).

TABLE 1
Symptom score evaluation prompts

| Region | Symptom Question |
|---|---|
| Nose | Runny nose (Q1), congested or stuffy nose (Q2), sinus pressure (Q17), |
| | sneezing times (Q36), and abnormal sense of smell or taste (Q33). |
| Throat | Scratchy or itchy throat (Q3), sore or painful throat (Q4), swollen throat |
| | (Q5), and difficulty swallowing (Q6). |
| Eyes | Teary or water eyes (Q7), sore or painful eyes (Q8), and eyes sensitive to |
| | light (Q9). |
| Chest/Respiratory | Trouble breathing (Q10), chest congestion (Q11), chest tightness (Q12), |
| | dry or hacking cough (Q13), wet or loose cough (Q14), coughing times |
| | (Q37), and coughed up mucus or phlegm (Q38). |
| GI | Lack of appetite (Q20), stomach ache (Q22), how many times did you |
| | vomit (Q34), and how many times did you have diarrhea (Q35). |
| Body/Systemic | Headache (Q15), head congestion (Q16), felt dizzy (Q18), felt lightheaded |
| | (Q19), felt nauseated (Q21), sleeping more than usual (Q23), difficulty |
| | staying asleep (Q24), difficulty falling asleep (Q25), body aches or pains |
| | (Q26), weak or tired (Q27), chills or shivering (Q28), felt cold (Q29), felt |
| | hot (Q30), sweating (Q31), and felt uncomfortable (Q32) |
| Smell/Taste | Abnormal sense of smell or taste (Q33). |

### Example 1: Viral Load

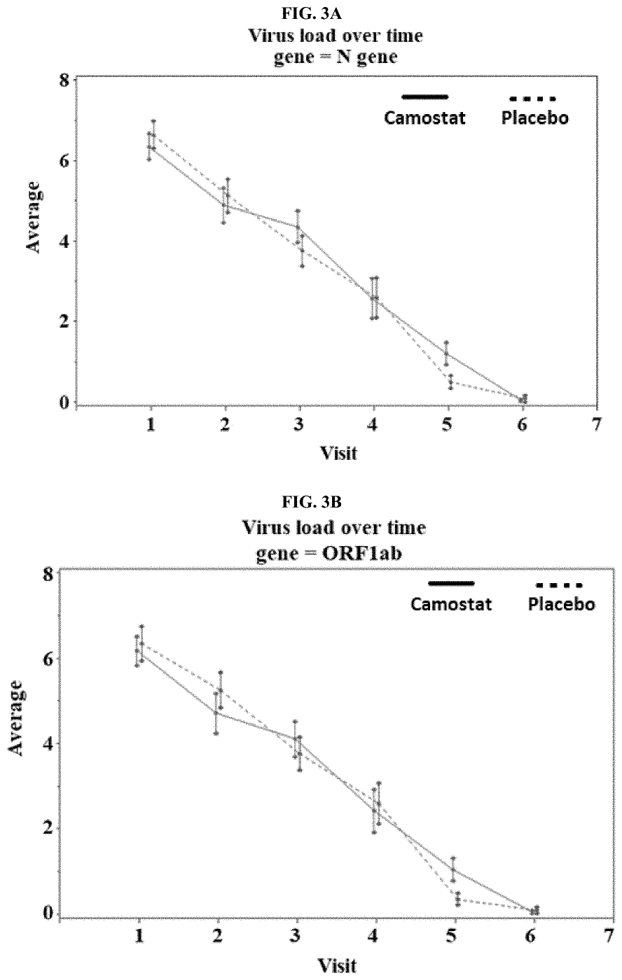

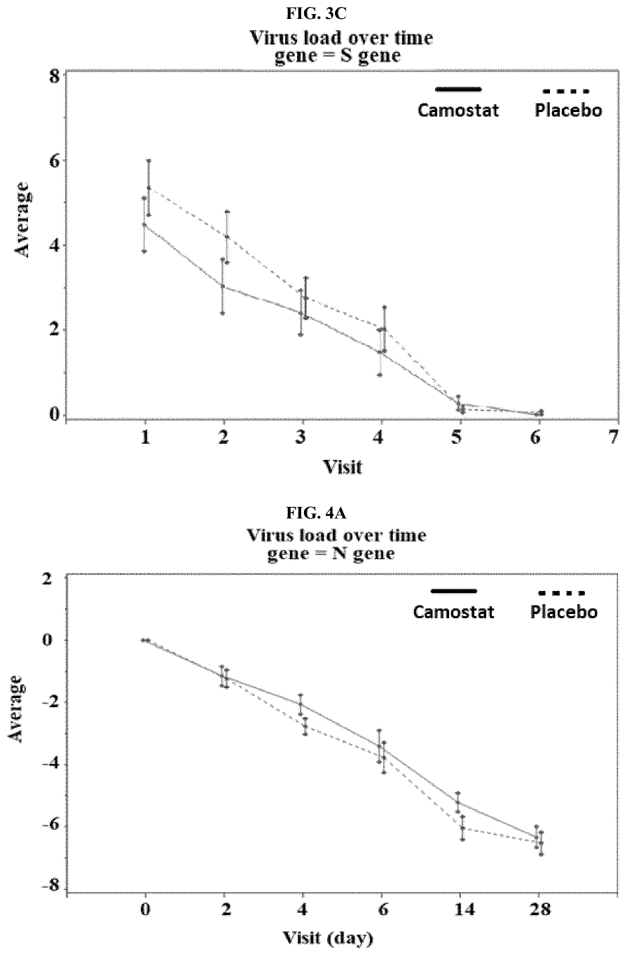

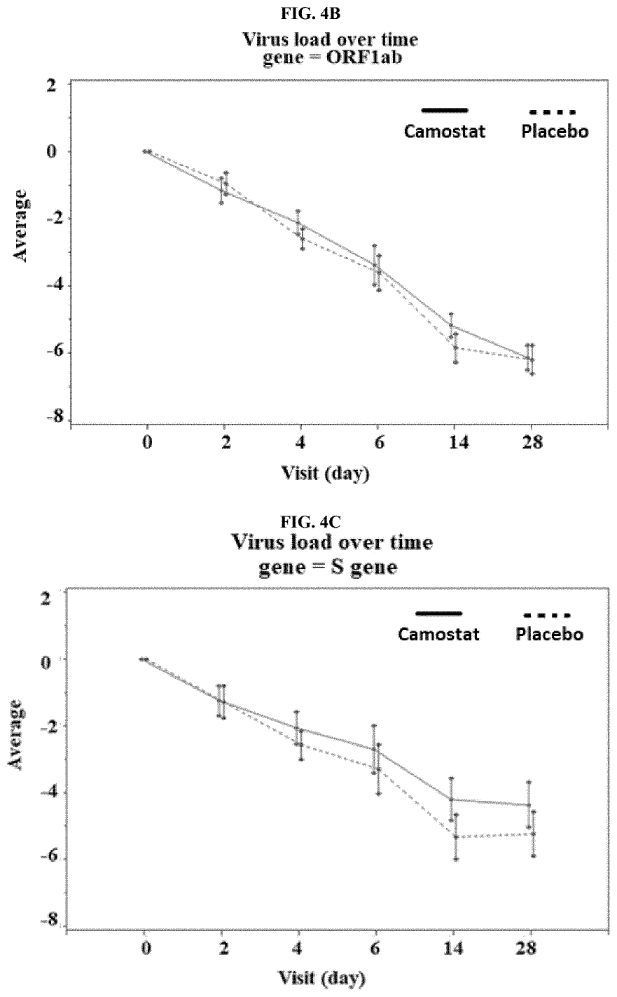

Raw means of log₁₀ viral load data obtained by RT-PCR are provided in Table 2 and FIGS. 3A-3C. Mean log₁₀ viral load decreased over time and was very low by day 28 (i.e., visit 6). Baseline viral load was higher in placebo compared to camostat. Changes in viral load from baseline are shown in Table 3 and FIGS. 4A-4C. Reductions in log₁₀ viral load from baseline to day 4 were greater in placebo.

TABLE 2
Analysis variable: viral load (log₁₀)

| | | | | | Std Dev | | | |
|---|---|---|---|---|---|---|---|---|
| Gene | Treatment | Visit | N | Mean | (S.D.) | Median | Min | Max |
| N | camostat | 1 | 35 | 6.35 | 1.89 | 6.47 | 1.55 | 8.90 |
| N | camostat | 2 | 25 | 4.89 | 2.19 | 4.49 | 0.00 | 8.74 |
| N | camostat | 3 | 33 | 4.35 | 2.27 | 4.56 | 0.00 | 8.14 |
| N | camostat | 4 | 22 | 2.58 | 2.31 | 2.48 | 0.00 | 6.84 |
| N | camostat | 5 | 33 | 1.21 | 1.58 | 0.12 | 0.00 | 4.95 |
| N | camostat | 6 | 32 | 0.05 | 0.15 | 0.00 | 0.00 | 0.66 |
| N | placebo | 1 | 34 | 6.64 | 1.99 | 7.31 | 0.00 | 8.70 |
| N | placebo | 2 | 28 | 5.13 | 2.17 | 5.69 | 0.00 | 8.29 |
| N | placebo | 3 | 33 | 3.76 | 2.17 | 3.79 | 0.00 | 7.57 |
| N | placebo | 4 | 25 | 2.59 | 2.48 | 2.50 | 0.00 | 8.13 |
| N | placebo | 5 | 33 | 0.49 | 0.89 | 0.00 | 0.00 | 3.07 |
| N | placebo | 6 | 34 | 0.08 | 0.45 | 0.00 | 0.00 | 2.64 |
| ORF1ab | camostat | 1 | 35 | 6.17 | 2.02 | 6.50 | 0.00 | 9.00 |
| ORF1ab | camostat | 2 | 25 | 4.71 | 2.33 | 4.20 | 0.00 | 9.29 |
| ORF1ab | camostat | 3 | 33 | 4.10 | 2.41 | 4.41 | 0.00 | 8.12 |
| ORF1ab | camostat | 4 | 22 | 2.42 | 2.37 | 2.47 | 0.00 | 7.33 |
| ORF1ab | camostat | 5 | 33 | 1.04 | 1.53 | 0.00 | 0.00 | 5.10 |
| ORF1ab | camostat | 6 | 32 | 0.05 | 0.22 | 0.00 | 0.00 | 1.12 |
| ORF1ab | placebo | 1 | 34 | 6.34 | 2.31 | 7.07 | 0.00 | 8.97 |
| ORF1ab | placebo | 2 | 28 | 5.25 | 2.21 | 5.81 | 0.00 | 8.06 |
| ORF1ab | placebo | 3 | 33 | 3.76 | 2.21 | 3.73 | 0.00 | 7.61 |
| ORF1ab | placebo | 4 | 25 | 2.60 | 2.39 | 2.46 | 0.00 | 7.17 |
| ORF1ab | placebo | 5 | 33 | 0.34 | 0.80 | 0.00 | 0.00 | 3.02 |
| ORF1ab | placebo | 6 | 34 | 0.09 | 0.42 | 0.00 | 0.00 | 2.40 |
| S | camostat | 1 | 35 | 4.50 | 3.69 | 5.02 | 0.00 | 12.39 |
| S | camostat | 2 | 25 | 3.04 | 3.13 | 3.22 | 0.00 | 11.68 |
| S | camostat | 3 | 33 | 2.42 | 2.96 | 1.01 | 0.00 | 10.03 |
| S | camostat | 4 | 22 | 1.48 | 2.52 | 0.00 | 0.00 | 8.72 |
| S | camostat | 5 | 33 | 0.27 | 0.98 | 0.00 | 0.00 | 4.99 |
| S | camostat | 6 | 32 | 0.00 | 0.00 | 0.00 | 0.00 | 0.00 |
| S | placebo | 1 | 34 | 5.37 | 3.82 | 6.44 | 0.00 | 12.12 |
| S | placebo | 2 | 28 | 4.20 | 3.17 | 5.21 | 0.00 | 10.70 |
| S | placebo | 3 | 33 | 2.76 | 2.67 | 2.55 | 0.00 | 8.69 |
| S | placebo | 4 | 25 | 2.02 | 2.56 | 0.00 | 0.00 | 7.80 |
| S | placebo | 5 | 33 | 0.12 | 0.45 | 0.00 | 0.00 | 2.37 |
| S | placebo | 6 | 34 | 0.04 | 0.25 | 0.00 | 0.00 | 1.47 |

TABLE 3
Analysis variable: change

| Gene | Treatment | Visit | N | Mean | S.D. | Median | L.Q. | U.Q. | Min | Max |
|---|---|---|---|---|---|---|---|---|---|---|
| N | camostat | 1 | 35 | 0.00 | 0.00 | 0.00 | 0.00 | 0.00 | 0.00 | 0.00 |
| N | camostat | 2 | 25 | −1.15 | 1.54 | −1.14 | −1.80 | −0.45 | −3.90 | 3.58 |
| N | camostat | 3 | 33 | −2.07 | 1.79 | −2.32 | −2.89 | −1.10 | −5.82 | 5.03 |
| N | camostat | 4 | 22 | −3.41 | 2.40 | −3.07 | −4.90 | −2.38 | −7.54 | 4.01 |
| N | camostat | 5 | 33 | −5.21 | 1.74 | −5.52 | −5.97 | −4.22 | −8.55 | −1.55 |
| N | camostat | 6 | 32 | −6.33 | 1.92 | −6.60 | −7.73 | −5.52 | −8.90 | −1.55 |
| N | placebo | 1 | 34 | 0.00 | 0.00 | 0.00 | 0.00 | 0.00 | 0.00 | 0.00 |
| N | placebo | 2 | 27 | −1.24 | 1.42 | −1.39 | −1.98 | −0.41 | −3.94 | 1.99 |
| N | placebo | 3 | 32 | −2.77 | 1.44 | −2.85 | −3.93 | −1.68 | −5.64 | 0.00 |
| N | placebo | 4 | 24 | −3.78 | 2.35 | −3.88 | −5.16 | −1.93 | −8.55 | 0.39 |
| N | placebo | 5 | 32 | −6.04 | 2.10 | −6.21 | −7.70 | −4.95 | −8.55 | 0.00 |
| N | placebo | 6 | 33 | −6.53 | 2.07 | −7.16 | −7.88 | −5.76 | −8.70 | 0.00 |
| ORF1ab | camostat | 1 | 35 | 0.00 | 0.00 | 0.00 | 0.00 | 0.00 | 0.00 | 0.00 |
| ORF1ab | camostat | 2 | 25 | −1.17 | 1.83 | −1.04 | −1.96 | −0.39 | −4.72 | 4.32 |
| ORF1ab | camostat | 3 | 33 | −2.14 | 2.00 | −2.33 | −3.17 | −1.07 | −5.67 | 5.80 |
| ORF1ab | camostat | 4 | 22 | −3.39 | 2.76 | −3.41 | −5.61 | −2.33 | −7.86 | 5.02 |
| ORF1ab | camostat | 5 | 33 | −5.19 | 1.95 | −5.61 | −6.50 | −3.91 | −8.28 | 0.00 |
| ORF1ab | camostat | 6 | 32 | −6.15 | 2.09 | −6.57 | −7.79 | −5.14 | −9.00 | 0.00 |
| ORF1ab | placebo | 1 | 34 | 0.00 | 0.00 | 0.00 | 0.00 | 0.00 | 0.00 | 0.00 |
| ORF1ab | placebo | 2 | 27 | −0.96 | 1.69 | −1.24 | −1.80 | 0.01 | −3.91 | 4.10 |
| ORF1ab | placebo | 3 | 32 | −2.61 | 1.70 | −2.77 | −3.82 | −1.56 | −6.21 | 2.20 |
| ORF1ab | placebo | 4 | 24 | −3.63 | 2.54 | −3.85 | −5.63 | −1.68 | −7.85 | 1.91 |
| ORF1ab | placebo | 5 | 32 | −5.87 | 2.38 | −6.57 | −7.77 | −4.43 | −8.79 | 0.00 |
| ORF1ab | placebo | 6 | 33 | −6.22 | 2.41 | −7.00 | −7.88 | −5.45 | −8.97 | 0.00 |
| S | camostat | 1 | 35 | 0.00 | 0.00 | 0.00 | 0.00 | 0.00 | 0.00 | 0.00 |
| S | camostat | 2 | 25 | −1.24 | 2.26 | −0.89 | −2.05 | 0.00 | −6.40 | 5.18 |
| S | camostat | 3 | 33 | −2.06 | 2.80 | −1.94 | −4.15 | 0.00 | −7.35 | 7.55 |
| S | camostat | 4 | 22 | −2.70 | 3.32 | −2.41 | −6.27 | 0.00 | −8.76 | 4.89 |
| S | camostat | 5 | 33 | −4.20 | 3.58 | −4.28 | −6.97 | 0.00 | −12.33 | 0.00 |
| S | camostat | 6 | 32 | −4.37 | 3.81 | −4.88 | −7.33 | 0.00 | −12.39 | 0.00 |
| S | placebo | 1 | 34 | 0.00 | 0.00 | 0.00 | 0.00 | 0.00 | 0.00 | 0.00 |
| S | placebo | 2 | 27 | −1.28 | 2.47 | −1.20 | −2.88 | 0.00 | −7.66 | 3.56 |
| S | placebo | 3 | 32 | −2.57 | 2.38 | −2.59 | −4.26 | 0.00 | −8.54 | 1.56 |
| S | placebo | 4 | 24 | −3.30 | 3.57 | −2.88 | −5.24 | 0.00 | −12.12 | 1.00 |
| S | placebo | 5 | 32 | −5.33 | 3.74 | −6.30 | −8.33 | −1.12 | −12.12 | 0.00 |
| S | placebo | 6 | 33 | −5.24 | 3.84 | −6.02 | −8.22 | 0.00 | −12.12 | 0.00 |

*LQ is lower quartile; UQ is upper quartile.

Comparison of treatment groups was performed using an ANCOVA model (adjusted for baseline viral load) implemented in a linear mixed model using all available subjects (n=57) and log₁₀ viral load from days 0-6. A reduction of −1.94 log₁₀ in camostat and −2.63 log₁₀ was in placebo was observed (Table 4). Thus, the difference between treatment groups in the changes from baseline to day 4 was −0.69 (SE=0.45) indicating a smaller reduction in camostat compared to placebo. The t-statistic for this comparison was −1.53 with a p-value of 0.13. This value crosses the predefined boundary of −1.00 for futility. Conditional power for the hypothesized effect, calculated using the B-method (Lan and Wittes, 1988), is 2.6%.

TABLE 4
ANCOVA using linear mixed model to compare differences
in viral load changes between treatment groups (n = 57)

| | Camostat | Placebo | | | |
|---|---|---|---|---|---|
| Visit | LSMean* | LSMean | Difference | T- | p- |
| (day) | Change (SE) | Change (SE) | (SE) | Statistic | value |
| 2 (2) | −1.10 (0.30) | −1.11 (0.30) | −0.01 (0.42) | −0.03 | 0.97 |
| 3 (4) | −1.94 (0.32) | −2.63 (0.32) | −0.69 (0.45) | −1.53 | 0.13 |
| 4 (6) | −3.22 (0.46) | −3.61 (0.45) | −0.39 (0.63) | −0.62 | 0.54 |

*LSMean—Least Squares Mean from repeated measures linear mixed model.

Sensitivity analyses indicate that these differences were consistent across analytic methods. Regardless of the chosen analytic model, the test statistic crosses the futility boundary.

Infectious viral load has not be assessed, but rather only PCR-detectable viral genomes in nasopharyngeal swabs. Thus, the effect of camostat vs. placebo on lower respiratory tract viral replication is not presently known.

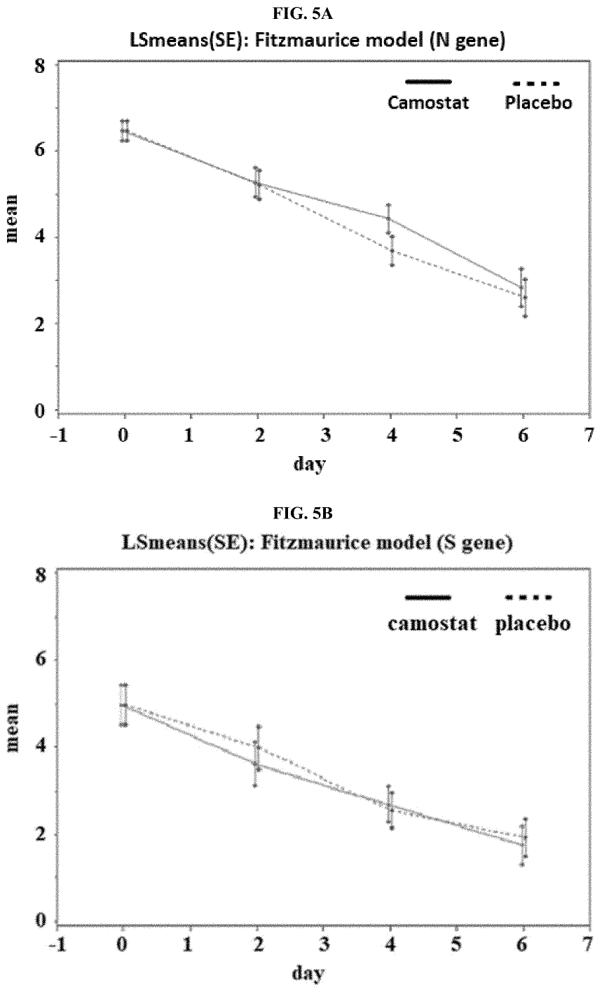

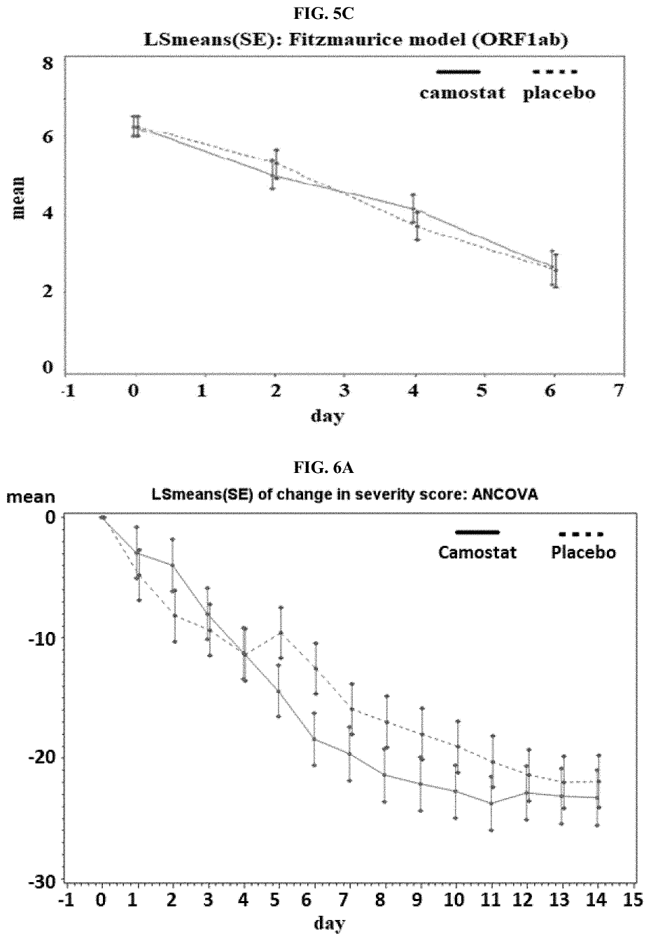

TABLE 5
Fitzmaurice mixed model controlling for baseline viral load (FIGS. 5A-5C)

| | | | | | |
|---|---|---|---|---|---|
| Label | Day | Camostat | Placebo | Effect | P value |

N gene

| | | | | | |
|---|---|---|---|---|---|
| Common baseline | 0 | 6.5 (6, 6.9) | 6.5 (6, 6.9) | — | — |
| A: visit 2 | 2 | 5.3 (4.6, 5.9) | 5.2 (4.6, 5.9) | — | — |
| A: visit 2 change | 2 | −1.2 (−1.8, −0.6) | −1.3 (−1.8, −0.7) | −0.06 (−0.83, 0.7) | 0.87 |
| A: visit 3(D4) | 4 | 4.4 (3.8, 5.1) | 3.7 (3, 4.3) | — | — |
| A: visit 3(D4) change | 4 | −2 (−2.6, −1.5) | −2.8 (−3.3, −2.2) | −0.74 (−1.51, 0.03) | 0.06 |
| A: visit 4(D6) | 6 | 2.8 (2, 3.7) | 2.6 (1.8, 3.4) | — | — |
| A: visit 4(D6) change | 6 | −3.6 (−4.5, −2.8) | −3.9 (−4.7, −3) | −0.23 (−1.4, 0.94) | 0.69 |

ORF1ab

| | | | | | |
|---|---|---|---|---|---|
| Common baseline | 0 | 6.3 (5.8, 6.8) | 6.3 (5.8, 6.8) | — | — |
| A: visit 2 | 2 | 5 (4.3, 5.8) | 5.3 (4.6, 6) | — | — |
| A: visit 2 change | 2 | −1.2 (−1.9, −0.6) | −1 (−1.6, −0.3) | 0.28 (−0.57, 1.13) | 0.52 |
| A: visit 3(D4) | 4 | 4.2 (3.5, 4.9) | 3.7 (3, 4.4) | — | — |
| A: visit 3(D4) change | 4 | −2.1 (−2.7, −1.5) | −2.6 (−3.2, −1.9) | −0.46 (−1.3, 0.39) | 0.29 |
| A: visit 4(D6) | 6 | 2.6 (1.8, 3.5) | 2.6 (1.7, 3.4) | — | — |
| A: visit 4(D6) change | 6 | −3.6 (−4.5, −2.7) | −3.7 (−4.6, −2.9) | −0.09 (−1.27, 1.08) | 0.87 |

S gene

| | | | | | |
|---|---|---|---|---|---|
| Common baseline | 0 | 5 (4.1, 5.9) | 5 (4.1, 5.9) | — | — |
| A: visit 2 | 2 | 3.6 (2.6, 4.6) | 4 (3, 5) | — | — |
| A: visit 2 change | 2 | −1.4 (−2.2, −0.5) | −1 (−1.8, −0.2) | 0.36 (−0.72, 1.43) | 0.51 |
| A: visit 3(D4) | 4 | 2.7 (1.9, 3.5) | 2.6 (1.7, 3.4) | — | — |
| A: visit 3(D4) change | 4 | −2.3 (−3.1, −1.5) | −2.4 (−3.2, −1.6) | −0.13 (−1.07, 0.8) | 0.77 |
| A: visit 4(D6) | 6 | 1.7 (0.8, 2.6) | 1.9 (1.1, 2.8) | — | — |
| A: visit 4(D6) change | 6 | −3.2 (−4.3, −2.2) | −3 (−4, −2) | 0.19 (−0.98, 1.36) | 0.75 |

### Example 2: COVID-19 Symptom Severity Score Evaluation

The COVID-19 PRO daily self-score tool consists of 39 items that are answered daily by the subject. Items 1-33 are Likert-scale questions (e.g., rated 0-4), wherein 0=not at all, and 4=very much. These items are summed to score the severity of symptoms, wherein a total score of 132 would indicate the greatest severity of symptoms and a score of 0 would indicate no severity of symptoms.

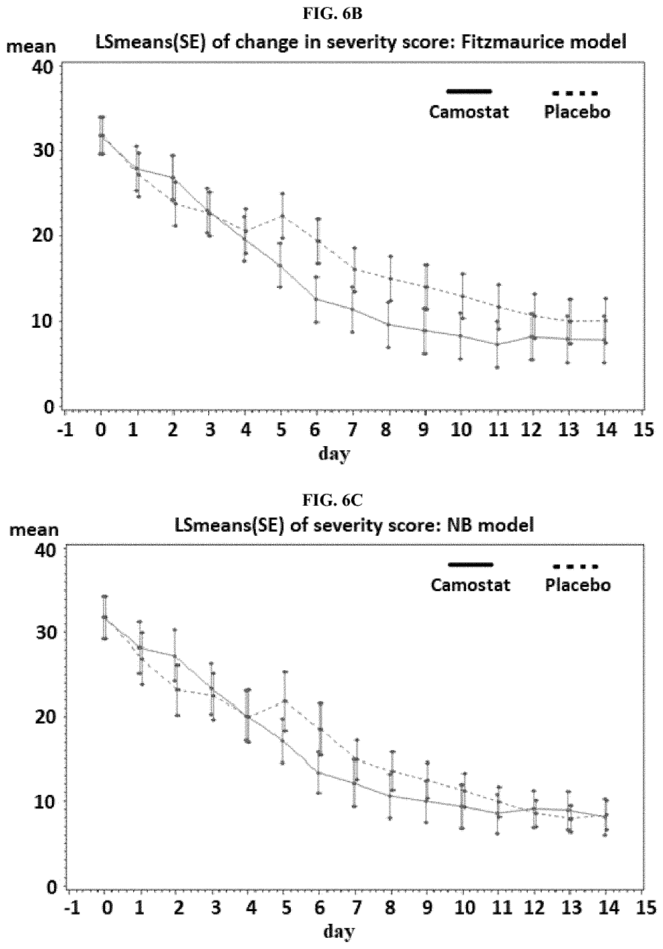

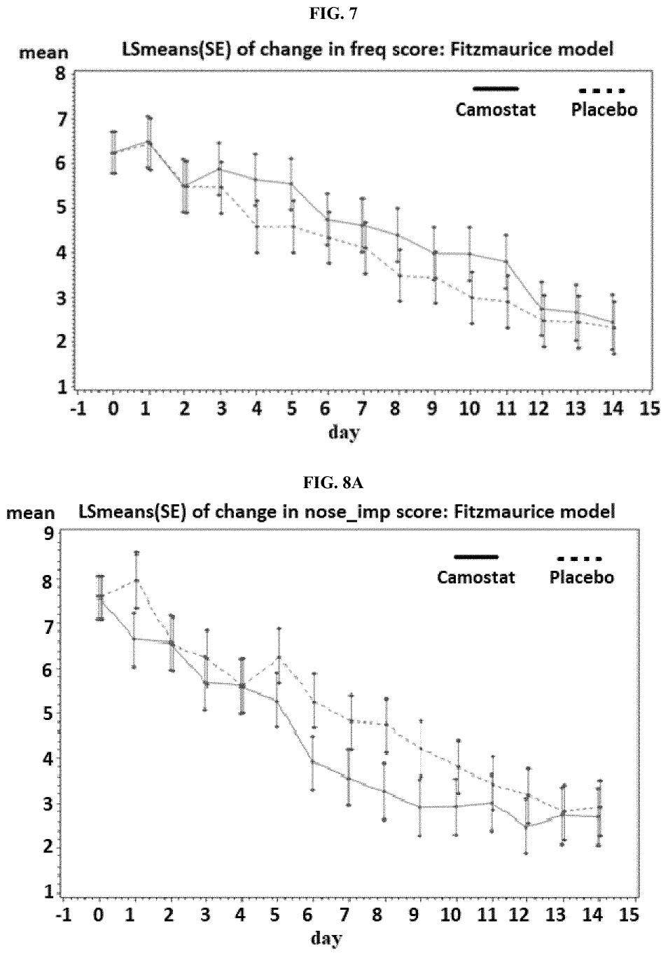

Raw means (SD) for COVID-19 symptom severity by treatment group and day are presented in Table 6. Results from the ANCOVA linear mixed model are shown in Table 7. Severity scores were lower at days 5 through 10 in camostat compared to placebo, but did not reach statistical significance (FIG. 6A-6C).

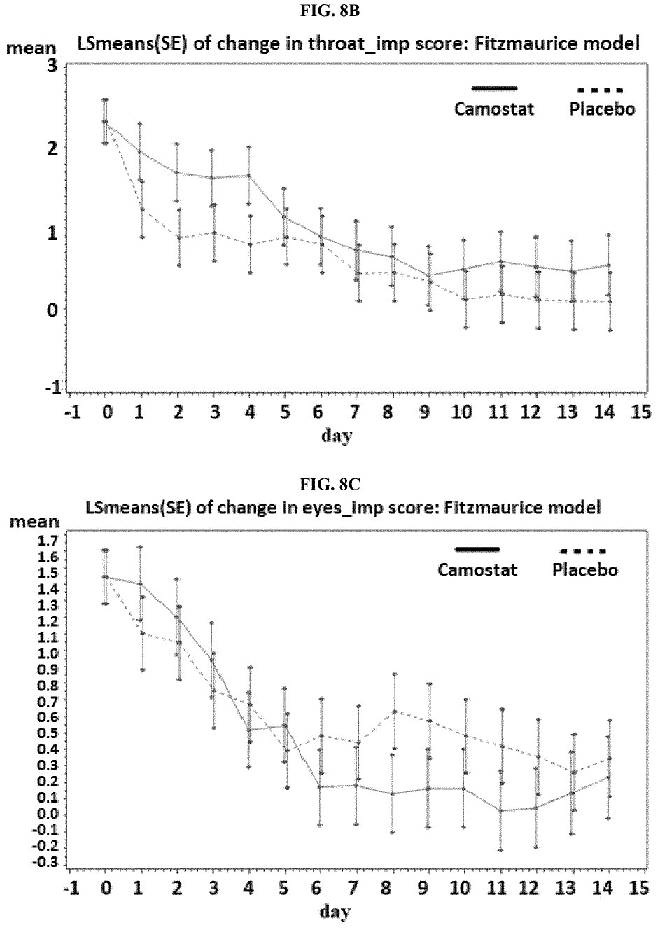

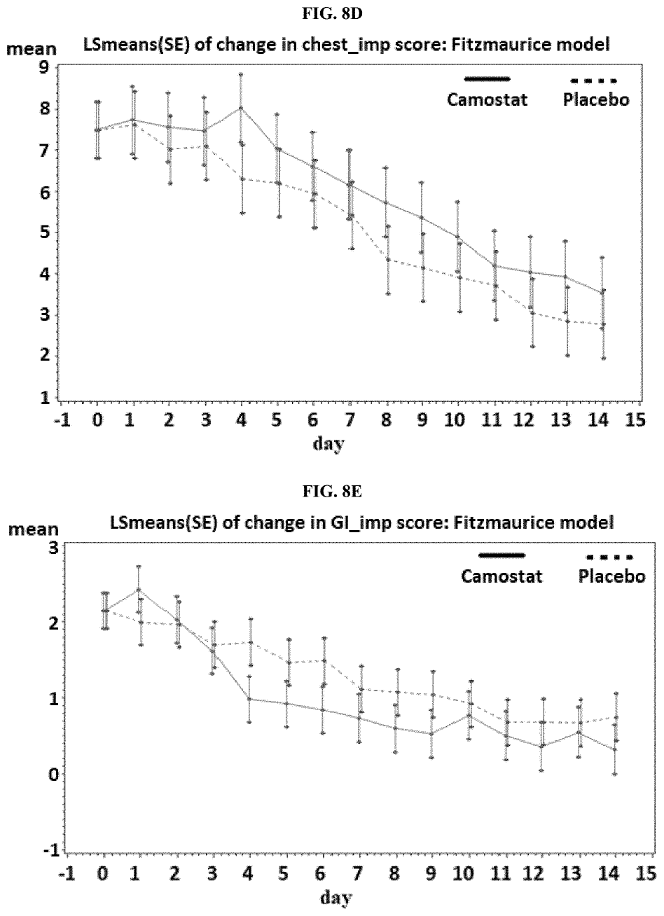

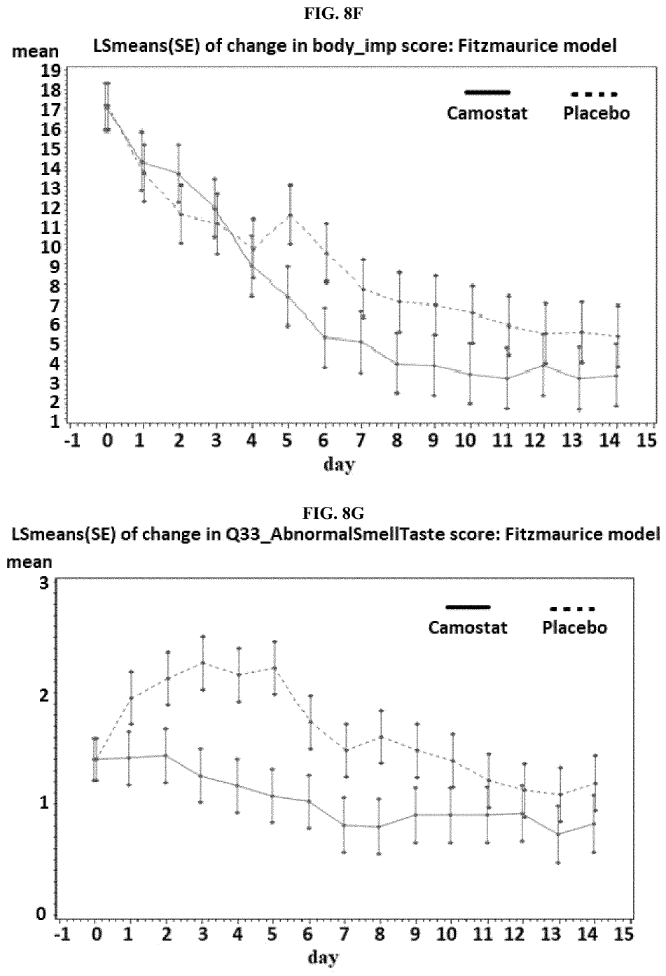

Additional statistical models were applied to the raw data, which are provided herein (Tables 8-10). Furthermore, analyses of particular families of symptoms (e.g. nose) were performed, and the results are additionally provided herein, demonstrating the utility of the method of the present disclosure (Tables 11-17, FIGS. 8A-8G).

TABLE 6
COVID-19 symptom severity score raw
means by treatment group and day

| | |
|---|---|
| Camostat | Placebo |

| Day | N | Mean | Std Dev | N | Mean | Std Dev |
|---|---|---|---|---|---|---|
| 0 | 35 | 34.9 | 22.7 | 35 | 28.7 | 20.2 |
| 1 | 34 | 30.4 | 23.3 | 34 | 25 | 21.9 |
| 2 | 31 | 29.7 | 23 | 34 | 21.6 | 20.7 |
| 3 | 33 | 24.1 | 21.1 | 33 | 19.8 | 17.6 |
| 4 | 33 | 20.8 | 20.3 | 32 | 17.6 | 20.6 |
| 5 | 33 | 17.7 | 18 | 33 | 20 | 22.5 |
| 6 | 32 | 14.1 | 16.4 | 33 | 17.3 | 20 |
| 7 | 29 | 12.6 | 17.9 | 34 | 13.8 | 15.5 |
| 8 | 29 | 11.4 | 16.5 | 33 | 12.1 | 14.4 |
| 9 | 29 | 10.7 | 15.3 | 33 | 11.1 | 13.4 |
| 10 | 29 | 10 | 15.7 | 33 | 10.1 | 13 |
| 11 | 28 | 9.3 | 14.3 | 33 | 8.8 | 11.4 |
| 12 | 28 | 9.6 | 13.9 | 32 | 7.5 | 9.7 |
| 13 | 25 | 9.7 | 14.2 | 31 | 6.9 | 9.8 |
| 14 | 25 | 8.2 | 13.1 | 30 | 7.5 | 11.1 |

TABLE 7
ANCOVA using linear mixed model to compare differences in
COVID-19 symptom severity score between treatment groups

| Day | A: Camostat arm | B: Placebo arm | B − A | P value |
|---|---|---|---|---|
| 1 | −2.93 (2.11) | −4.76 (2.1) | −1.83 (2.99) | 0.54 |
| 2 | −3.96 (2.16) | −8.18 (2.1) | −4.22 (3.02) | 0.16 |
| 3 | −8.01 (2.13) | −9.35 (2.12) | −1.34 (3.01) | 0.66 |
| 4 | −11.29 (2.13) | −11.39 (2.13) | −0.1 (3.02) | 0.97 |
| 5 | −14.41 (2.13) | −9.58 (2.12) | 4.84 (3.01) | 0.11 |
| 6 | −18.43 (2.15) | −12.53 (2.12) | 5.89 (3.02) | 0.05 |
| 7 | −19.64 (2.2) | −15.92 (2.1) | 3.71 (3.05) | 0.22 |
| 8 | −21.42 (2.2) | −16.99 (2.12) | 4.43 (3.06) | 0.15 |
| 9 | −22.13 (2.2) | −17.98 (2.12) | 4.16 (3.06) | 0.18 |
| 10 | −22.75 (2.2) | −19.05 (2.12) | 3.7 (3.06) | 0.23 |
| 11 | −23.77 (2.22) | −20.28 (2.12) | 3.5 (3.08) | 0.26 |
| 12 | −22.87 (2.22) | −21.4 (2.13) | 1.46 (3.09) | 0.64 |
| 13 | −23.17 (2.29) | −22 (2.15) | 1.17 (3.15) | 0.71 |
| 14 | −23.26 (2.29) | −21.94 (2.17) | 1.32 (3.16) | 0.68 |

TABLE 8
Fitzmaurice mixed effects model of symptom score results

| Day | A: Camostat arm | B: Placebo arm | B − A | P value |
|---|---|---|---|---|
| 0 | 31.81 (2.17) | 31.81 (2.17) | 0 | |
| 1 | 27.95 (2.59) | 27.21 (2.59) | −0.73 (2.76) | 0.79 |
| 2 | 26.89 (2.63) | 23.8 (2.59) | −3.09 (2.8) | 0.27 |
| 3 | 22.97 (2.6) | 22.61 (2.6) | −0.36 (2.79) | 0.90 |
| 4 | 19.69 (2.6) | 20.58 (2.61) | 0.89 (2.8) | 0.75 |
| 5 | 16.57 (2.6) | 22.41 (2.6) | 5.84 (2.79) | 0.04 |
| 6 | 12.53 (2.62) | 19.43 (2.6) | 6.9 (2.81) | 0.014 |
| 7 | 11.37 (2.67) | 16.06 (2.59) | 4.68 (2.84) | 0.10 |
| 8 | 9.57 (2.67) | 14.98 (2.6) | 5.4 (2.85) | 0.06 |
| 9 | 8.86 (2.67) | 13.99 (2.6) | 5.13 (2.85) | 0.07 |
| 10 | 8.24 (2.67) | 12.91 (2.6) | 4.67 (2.85) | 0.10 |
| 11 | 7.25 (2.69) | 11.69 (2.6) | 4.44 (2.87) | 0.12 |
| 12 | 8.18 (2.69) | 10.56 (2.61) | 2.39 (2.88) | 0.41 |
| 13 | 7.87 (2.75) | 9.98 (2.63) | 2.11 (2.95) | 0.48 |
| 14 | 7.8 (2.75) | 10.05 (2.65) | 2.24 (2.97) | 0.45 |

TABLE 9
Negative binomial model

| Day | A: Camostat arm | B: Placebo arm | B − A | P value |
|---|---|---|---|---|
| 0 | 31.81 (2.56) | 31.81 (2.56) | 0 | — |
| 1 | 28.22 (3.06) | 26.87 (3.03) | 0.95 (0.1) | 0.65 |
| 2 | 27.28 (3.05) | 23.2 (2.94) | 0.85 (0.1) | 0.18 |
| 3 | 23.35 (3.02) | 22.43 (2.72) | 0.96 (0.13) | 0.77 |
| 4 | 20.17 (2.89) | 20.08 (3.08) | 1 (0.17) | 0.98 |
| 5 | 17.14 (2.63) | 21.84 (3.51) | 1.27 (0.24) | 0.20 |
| 6 | 13.4 (2.45) | 18.53 (3.05) | 1.38 (0.29) | 0.12 |
| 7 | 12.09 (2.79) | 14.88 (2.34) | 1.23 (0.3) | 0.39 |
| 8 | 10.6 (2.64) | 13.56 (2.24) | 1.28 (0.34) | 0.36 |
| 9 | 9.94 (2.43) | 12.45 (2.11) | 1.25 (0.33) | 0.40 |
| 10 | 9.36 (2.56) | 11.26 (2.05) | 1.2 (0.36) | 0.54 |
| 11 | 8.49 (2.3) | 9.89 (1.82) | 1.17 (0.35) | 0.61 |
| 12 | 9.06 (2.18) | 8.54 (1.52) | 0.94 (0.25) | 0.82 |
| 13 | 8.9 (2.27) | 7.86 (1.52) | 0.88 (0.26) | 0.67 |
| 14 | 8.11 (2.11) | 8.34 (1.71) | 1.03 (0.31) | 0.93 |

TABLE 10
Results of Fitzmaurice mixed effects model for frequency score

| Day | A: Camostat arm | B: Placebo arm | B − A | P value |
|---|---|---|---|---|
| 0 | 6.25 (0.47) | 6.25 (0.47) | 0 | — |
| 1 | 6.49 (0.57) | 6.44 (0.57) | −0.05 (0.67) | 0.94 |
| 2 | 5.51 (0.59) | 5.48 (0.57) | −0.03 (0.68) | 0.97 |
| 3 | 5.88 (0.58) | 5.46 (0.57) | −0.42 (0.67) | 0.54 |
| 4 | 5.64 (0.58) | 4.58 (0.58) | −1.05 (0.68) | 0.12 |
| 5 | 5.54 (0.58) | 4.59 (0.58) | −0.96 (0.68) | 0.16 |
| 6 | 4.75 (0.58) | 4.34 (0.57) | −0.41 (0.68) | 0.54 |
| 7 | 4.62 (0.59) | 4.11 (0.57) | −0.51 (0.68) | 0.46 |
| 8 | 4.39 (0.6) | 3.49 (0.58) | −0.9 (0.7) | 0.19 |
| 9 | 3.98 (0.59) | 3.44 (0.57) | −0.54 (0.69) | 0.43 |
| 10 | 3.96 (0.6) | 2.99 (0.57) | −0.98 (0.69) | 0.16 |
| 11 | 3.8 (0.6) | 2.9 (0.58) | −0.9 (0.7) | 0.20 |
| 12 | 2.75 (0.6) | 2.47 (0.58) | −0.28 (0.7) | 0.69 |
| 13 | 2.66 (0.62) | 2.44 (0.58) | −0.22 (0.72) | 0.76 |
| 14 | 2.44 (0.61) | 2.32(0.59) | −0.13 (0.72) | 0.86 |

TABLE 11
Results of Fitzmaurice mixed effects
model for nose-family symptoms

| | | | | | Proba- |
|---|---|---|---|---|---|
| Day | DayPRO | Camostat | Placebo | Effect | bility |
| 0 | 0 | 7.6 (0.5) | 7.6 (0.5) | — | — |
| 1 | 1 | 6.6 (0.6) | 8 (0.6) | 1.3 (0.7) | 0.0589 |
| 2 | 2 | 6.6 (0.6) | 6.5 (0.6) | 0 (0.7) | 0.9677 |
| 3 | 3 | 5.7 (0.6) | 6.2 (0.6) | 0.5 (0.7) | 0.4416 |
| 4 | 4 | 5.6 (0.6) | 5.6 (0.6) | 0 (0.7) | 0.9891 |
| 5 | 5 | 5.3 (0.6) | 6.3 (0.6) | 1 (0.7) | 0.1679 |
| 6 | 6 | 3.9 (0.6) | 5.3 (0.6) | 1.4 (0.7) | 0.0546 |
| 7 | 7 | 3.6 (0.6) | 4.8 (0.6) | 1.2 (0.7) | 0.0865 |
| 8 | 8 | 3.3 (0.6) | 4.7 (0.6) | 1.5 (0.7) | 0.0407 |
| 9 | 9 | 2.9 (0.6) | 4.2 (0.6) | 1.3 (0.7) | 0.0717 |
| 10 | 10 | 2.9 (0.6) | 3.8 (0.6) | 0.9 (0.7) | 0.2219 |
| 11 | 11 | 3 (0.6) | 3.4 (0.6) | 0.4 (0.7) | 0.5687 |
| 12 | 12 | 2.5 (0.6) | 3.2 (0.6) | 0.7 (0.7) | 0.3461 |
| 13 | 13 | 2.7 (0.6) | 2.8 (0.6) | 0.1 (0.7) | 0.9104 |
| 14 | 14 | 2.7 (0.6) | 2.9 (0.6) | 0.2 (0.8) | 0.7776 |

TABLE 12
Results of Fitzmaurice mixed effects
model for throat-family symptoms

| | | | | | Proba- |
|---|---|---|---|---|---|
| Day | DayPRO | Camostat | Placebo | Effect | bility |
| 0 | 0 | 2.3 (0.3) | 2.3 (0.3) | — | — |
| 1 | 1 | 2 (0.3) | 1.2 (0.3) | −0.7 (0.4) | 0.0917 |
| 2 | 2 | 1.7 (0.4) | 0.9 (0.3) | −0.8 (0.4) | 0.0617 |
| 3 | 3 | 1.6 (0.3) | 0.9 (0.3) | −0.7 (0.4) | 0.1133 |
| 4 | 4 | 1.7 (0.3) | 0.8 (0.4) | −0.9 (0.4) | 0.0482 |
| 5 | 5 | 1.1 (0.3) | 0.9 (0.3) | −0.2 (0.4) | 0.5672 |
| 6 | 6 | 0.9 (0.4) | 0.8 (0.3) | −0.1 (0.4) | 0.8258 |
| 7 | 7 | 0.7 (0.4) | 0.4 (0.3) | −0.3 (0.4) | 0.5185 |
| 8 | 8 | 0.6 (0.4) | 0.4 (0.3) | −0.2 (0.4) | 0.6515 |
| 9 | 9 | 0.4 (0.4) | 0.3 (0.3) | −0.1 (0.4) | 0.8579 |
| 10 | 10 | 0.5 (0.4) | 0.1 (0.3) | −0.4 (0.4) | 0.3994 |
| 11 | 11 | 0.6 (0.4) | 0.2 (0.3) | −0.4 (0.4) | 0.3602 |
| 12 | 12 | 0.5 (0.4) | 0.1 (0.4) | −0.4 (0.4) | 0.3513 |
| 13 | 13 | 0.5 (0.4) | 0.1 (0.4) | −0.4 (0.5) | 0.4211 |
| 14 | 14 | 0.5 (0.4) | 0.1 (0.4) | −0.5 (0.5) | 0.3235 |

TABLE 13
Results of Fitzmaurice mixed effects
model for eye-family symptoms

| | | | | | Proba- |
|---|---|---|---|---|---|
| Day | DayPRO | Camostat | Placebo | Effect | bility |
| 0 | 0 | 1.4 (0.2) | 1.4 (0.2) | — | — |
| 1 | 1 | 1.4 (0.2) | 1.1 (0.2) | −0.3 (0.3) | 0.3109 |
| 2 | 2 | 1.2 (0.2) | 1 (0.2) | −0.2 (0.3) | 0.6005 |
| 3 | 3 | 0.9 (0.2) | 0.8 (0.2) | −0.2 (0.3) | 0.5432 |
| 4 | 4 | 0.5 (0.2) | 0.7 (0.2) | 0.2 (0.3) | 0.6101 |
| 5 | 5 | 0.5 (0.2) | 0.4 (0.2) | −0.2 (0.3) | 0.6097 |
| 6 | 6 | 0.2 (0.2) | 0.5 (0.2) | 0.3 (0.3) | 0.3043 |
| 7 | 7 | 0.2 (0.2) | 0.4 (0.2) | 0.3 (0.3) | 0.3994 |
| 8 | 8 | 0.1 (0.2) | 0.6 (0.2) | 0.5 (0.3) | 0.1052 |
| 9 | 9 | 0.2 (0.2) | 0.6 (0.2) | 0.4 (0.3) | 0.1890 |
| 10 | 10 | 0.2 (0.2) | 0.5 (0.2) | 0.3 (0.3) | 0.3077 |
| 11 | 11 | 0 (0.2) | 0.4 (0.2) | 0.4 (0.3) | 0.2090 |
| 12 | 12 | 0 (0.2) | 0.4 (0.2) | 0.3 (0.3) | 0.3251 |
| 13 | 13 | 0.1 (0.2) | 0.3 (0.2) | 0.1 (0.3) | 0.7014 |
| 14 | 14 | 0.2 (0.2) | 0.3 (0.2) | 0.1 (0.3) | 0.7180 |

TABLE 14
Results of Fitzmaurice mixed effects model
for chest/respiratory-family symptoms

| | | | | | Proba- |
|---|---|---|---|---|---|
| Day | DayPRO | Camostat | Placebo | Effect | bility |
| 0 | 0 | 7.5 (0.7) | 7.5 (0.7) | — | — |
| 1 | 1 | 7.7 (0.8) | 7.6 (0.8) | −0.1 (0.9) | 0.8868 |
| 2 | 2 | 7.6 (0.8) | 7 (0.8) | −0.5 (0.9) | 0.5416 |
| 3 | 3 | 7.5 (0.8) | 7.1 (0.8) | −0.4 (0.9) | 0.6778 |
| 4 | 4 | 8 (0.8) | 6.3 (0.8) | −1.7 (0.9) | 0.0520 |
| 5 | 5 | 7 (0.8) | 6.2 (0.8) | −0.8 (0.9) | 0.3361 |
| 6 | 6 | 6.6 (0.8) | 5.9 (0.8) | −0.7 (0.9) | 0.4502 |
| 7 | 7 | 6.2 (0.8) | 5.4 (0.8) | −0.7 (0.9) | 0.4019 |
| 8 | 8 | 5.7 (0.8) | 4.3 (0.8) | −1.4 (0.9) | 0.1195 |
| 9 | 9 | 5.4 (0.8) | 4.2 (0.8) | −1.2 (0.9) | 0.1737 |
| 10 | 10 | 4.9 (0.8) | 3.9 (0.8) | −1 (0.9) | 0.2691 |
| 11 | 11 | 4.2 (0.8) | 3.7 (0.8) | −0.5 (0.9) | 0.5897 |
| 12 | 12 | 4 (0.8) | 3.1 (0.8) | −1 (0.9) | 0.2738 |
| 13 | 13 | 3.9 (0.9) | 2.9 (0.8) | −1.1 (0.9) | 0.2471 |
| 14 | 14 | 3.5 (0.9) | 2.8 (0.8) | −0.8 (0.9) | 0.4172 |

TABLE 15
Results of Fitzmaurice mixed effects model
for gastrointestinal-family symptoms

| | | | | | Proba- |
|---|---|---|---|---|---|
| Day | DayPRO | Camostat | Placebo | Effect | bility |
| 0 | 0 | 2.1 (0.2) | 2.1 (0.2) | — | — |
| 1 | 1 | 2.4 (0.3) | 2 (0.3) | −0.4 (0.4) | 0.2424 |
| 2 | 2 | 2 (0.3) | 2 (0.3) | −0.1 (0.4) | 0.8632 |
| 3 | 3 | 1.6 (0.3) | 1.7 (0.3) | 0.1 (0.4) | 0.8310 |
| 4 | 4 | 1 (0.3) | 1.7 (0.3) | 0.8 (0.4) | 0.0454 |
| 5 | 5 | 0.9 (0.3) | 1.5 (0.3) | 0.5 (0.4) | 0.1444 |
| 6 | 6 | 0.8 (0.3) | 1.5 (0.3) | 0.6 (0.4) | 0.0853 |
| 7 | 7 | 0.7 (0.3) | 1.1 (0.3) | 0.4 (0.4) | 0.3141 |
| 8 | 8 | 0.6 (0.3) | 1.1 (0.3) | 0.5 (0.4) | 0.2124 |
| 9 | 9 | 0.5 (0.3) | 1 (0.3) | 0.5 (0.4) | 0.1777 |
| 10 | 10 | 0.8 (0.3) | 0.9 (0.3) | 0.2 (0.4) | 0.6899 |
| 11 | 11 | 0.5 (0.3) | 0.7 (0.3) | 0.2 (0.4) | 0.6500 |
| 12 | 12 | 0.4 (0.3) | 0.7 (0.3) | 0.3 (0.4) | 0.4022 |
| 13 | 13 | 0.5 (0.3) | 0.7 (0.3) | 0.1 (0.4) | 0.7590 |
| 14 | 14 | 0.3 (0.3) | 0.7 (0.3) | 0.4 (0.4) | 0.2835 |

TABLE 16
Results of Fitzmaurice mixed effects
model for body-family symptoms

| | | | | | Proba- |
|---|---|---|---|---|---|
| Day | DayPRO | Camostat | Placebo | Effect | bility |
| 0 | 0 | 17.1 (1.2) | 17.1 (1.2) | — | — |
| 1 | 1 | 14.3 (1.5) | 13.7 (1.5) | −0.6 (1.7) | 0.7174 |
| 2 | 2 | 13.6 (1.5) | 11.5 (1.5) | −2.1 (1.7) | 0.2244 |
| 3 | 3 | 11.9 (1.5) | 11.1 (1.5) | −0.8 (1.7) | 0.6385 |
| 4 | 4 | 8.9 (1.5) | 9.8 (1.5) | 0.9 (1.7) | 0.5960 |
| 5 | 5 | 7.3 (1.5) | 11.5 (1.5) | 4.2 (1.7) | 0.0146 |
| 6 | 6 | 5.2 (1.5) | 9.6 (1.5) | 4.4 (1.7) | 0.0109 |
| 7 | 7 | 4.9 (1.6) | 7.7 (1.5) | 2.8 (1.7) | 0.1123 |
| 8 | 8 | 3.9 (1.6) | 7 (1.5) | 3.1 (1.8) | 0.0743 |
| 9 | 9 | 3.8 (1.6) | 6.9 (1.5) | 3.1 (1.8) | 0.0788 |
| 10 | 10 | 3.3 (1.6) | 6.4 (1.5) | 3.1 (1.8) | 0.0775 |
| 11 | 11 | 3 (1.6) | 5.8 (1.5) | 2.8 (1.8) | 0.1168 |
| 12 | 12 | 3.8 (1.6) | 5.4 (1.5) | 1.6 (1.8) | 0.3552 |
| 13 | 13 | 3 (1.6) | 5.5 (1.5) | 2.4 (1.8) | 0.1817 |
| 14 | 14 | 3.2 (1.6) | 5.3 (1.5) | 2 (1.8) | 0.2679 |

TABLE 17
Results of Fitzmaurice mixed effects
model for smell/taste-family symptoms

| | | | | | Proba- |
|---|---|---|---|---|---|
| Day | DayPRO | Camostat | Placebo | Effect | bility |
| 0 | 0 | 1.4 (0.2) | 1.4 (0.2) | | . |
| 1 | 1 | 1.4 (0.2) | 2 (0.2) | 0.5 (0.3) | 0.0564 |
| 2 | 2 | 1.4 (0.2) | 2.1 (0.2) | 0.7 (0.3) | 0.0157 |
| 3 | 3 | 1.3 (0.2) | 2.3 (0.2) | 1 (0.3) | 0.0004 |
| 4 | 4 | 1.2 (0.2) | 2.2 (0.2) | 1 (0.3) | 0.0005 |
| 5 | 5 | 1.1 (0.2) | 2.2 (0.2) | 1.2 (0.3) | <.0001 |
| 6 | 6 | 1 (0.2) | 1.7 (0.2) | 0.7 (0.3) | 0.0132 |
| 7 | 7 | 0.8 (0.2) | 1.5 (0.2) | 0.7 (0.3) | 0.0213 |
| 8 | 8 | 0.8 (0.2) | 1.6 (0.2) | 0.8 (0.3) | 0.0061 |
| 9 | 9 | 0.9 (0.2) | 1.5 (0.2) | 0.6 (0.3) | 0.0497 |
| 10 | 10 | 0.9 (0.2) | 1.4 (0.2) | 0.5 (0.3) | 0.0948 |
| 11 | 11 | 0.9 (0.2) | 1.2 (0.2) | 0.3 (0.3) | 0.2990 |
| 12 | 12 | 0.9 (0.2) | 1.1 (0.2) | 0.2 (0.3) | 0.4888 |
| 13 | 13 | 0.7 (0.3) | 1.1 (0.2) | 0.4 (0.3) | 0.2390 |
| 14 | 14 | 0.8 (0.3) | 1.2 (0.2) | 0.4 (0.3) | 0.2299 |

### ENUMERATED EMBODIMENTS

The following exemplary embodiments are provided, the numbering of which is not to be construed as designating levels of importance:

Embodiment 1 provides a method of treating, ameliorating, preventing, shortening duration, and/or reversing at least one symptom and/or complication in a human subject with a coronavirus infection, the method comprising administering to the subject a dose of about 645 mg of camostat free base, or an equimolar amount of a pharmaceutically acceptable salt or solvate thereof.

Embodiment 2 provides a method of preventing and/or reducing the occurrence of long COVID, and/or a symptom and/or complication thereof, in a human subject with a SARS-CoV-2 infection; hospitalization of a human subject with a SARS-CoV-2 infection; and/or death in a human subject with a SARS-CoV-2 infection; the method comprising administering to the subject a dose of about 645 mg of camostat free base, or an equimolar amount of a pharmaceutically acceptable salt or solvate thereof.

Embodiment 3 provides the method of Embodiment 1, wherein the coronavirus is at least one of MERS-CoV, SARS-CoV, and/or SARS-CoV-2.

Embodiment 4 provides the method of Embodiment 1 or 3, wherein the coronavirus is SARS-CoV-2.

Embodiment 5 provides the method of any one of Embodiments 1 and 3-4, wherein the subject suffers from long COVID.

Embodiment 6 provides the method of any one of Embodiments 1-5, wherein the administering reverses, reduces, and/or prevents progression of the coronavirus infection.

Embodiment 7 provides the method of Embodiment 6, wherein the progression of the coronavirus infection comprises hospitalization and/or death.

Embodiment 8 provides the method of any one of Embodiments 1-7, wherein the administering reduces, reverses, and/or eliminates at least one symptom of the coronavirus infection.

Embodiment 9 provides the method of any one of Embodiments 1-8, wherein the administering reduces recovery time for at least one symptom of the coronavirus infection.

Embodiment 10 provides the method of any one of Embodiments 1-9, wherein the at least one symptom is selected from the group consisting of runny nose, congested nose, sinus pressure, sneezing, scratchy or itchy throat, sore or painful throat, swollen throat, difficulty swallowing, teary or water eyes, sore or painful eyes, eyes sensitive to light, difficulty breathing, chest congestion, chest tightness, dry or hacking cough, wet or loose cough, frequent coughing, coughing mucus or phlegm, lack of appetite, gastrointestinal discomfort (i.e., stomachache), vomiting, diarrhea, headache, head congestion, dizziness, lightheadedness, nausea, dyspnea, myalgia, fever, excessive sleeping, difficulty sleeping, body aches or pains, fatigue, chills or shivering, feeling cold, feeling hot, sweating, discomfort, abnormal, reduced, or eliminated sense of smell (e.g., anosmia), and abnormal, reduced, or eliminated sense of taste (e.g., ageusia).

Embodiment 11 provides the method of any one of Embodiments 1-10, wherein the at least one symptom comprises anosmia.

Embodiment 12 provides the method of any one of Embodiments 1-10, wherein the at least one symptom comprises ageusia.

Embodiment 13 provides the method of any one of Embodiments 9-12, wherein the recovery time is reduced by at least about 1 to about 10 days as compared to a control human subject who is not administered the daily dose of about 645 mg of camostat free base, or an equimolar amount of a pharmaceutically acceptable salt or solvate thereof.

Embodiment 14 provides the method of Embodiment 13, wherein the recovery time is reduced by about 5 days as compared to a control human subject who is not administered the daily dose of about 645 mg of camostat free base, or an equimolar amount of a pharmaceutically acceptable salt or solvate thereof.

Embodiment 15 provides the method of any one of Embodiments 1-14, wherein the administration to the subject is by at least one route selected from the group consisting of nasal, inhalational, topical, oral, buccal, rectal, pleural, peritoneal, vaginal, intramuscular, subcutaneous, transdermal, epidural, intratracheal, otic, intraocular, intrathecal, and intravenous routes.

Embodiment 16 provides the method of Embodiment 15, wherein the administration to the subject is by an oral route.

Embodiment 17 provides the method of any one of Embodiments 1-16, wherein the administration of the dose to the subject is performed daily.

Embodiment 18 provides the method of any one of Embodiments 1-17, wherein the administration to the subject is performed for a period of 7 days.

Embodiment 19 provides the method of any one of Embodiments 17-18, wherein the daily administration comprises 800 mg of camostat mesylate.

Embodiment 20 provides the method of any one of Embodiments 17-18, wherein the daily administration comprises four equal doses of camostat free base, or a pharmaceutically acceptable salt or solvate thereof.

Embodiment 21 provides the method of Embodiment 20, wherein each of the four equal doses comprises about 200 mg of camostat mesylate.

Embodiment 22 provides the method of any one of Embodiments 20-21, wherein each dose is administered within about 3 hours of the previous and/or following dose.

Embodiment 23 provides the method of any one of Embodiments 20-21, wherein each dose is administered within about 4 hours of the previous and/or following dose. Embodiment 24 provides the method of any one of Embodiments 20-21, wherein each dose is administered within about 5 hours of the previous and/or following dose.

Embodiment 25 provides the method of any one of Embodiments 20-21, wherein each dose is administered within about 6 hours of the previous and/or following dose.

Embodiment 26 provides the method of any one of Embodiments 1-25, wherein the administration affords a trough plasma concentration of 4-(4-guanidinobenzoyloxy)phenylacetic acid (GBPA) in the subject of about 0.1 ng/ml to about 25 ng/ml.

Embodiment 27 provides the method of Embodiment 26, wherein the trough plasma concentration of GBPA in the subject is selected from the group consisting of about 0.1 ng/ml to about 5 ng/ml, about 5 ng/ml to about 10 ng/ml, about 10 ng/ml to about 15 ng/ml, about 15 ng/ml to about 20 ng/ml, and about 20 ng/ml to about 25 ng/ml.

Embodiment 28 provides the method of any one of Embodiments 1-27, wherein the administration affords an average plasma concentration of GBPA in the subject of about 0.09 μM to about 0.13 μM.

Embodiment 29 provides the method of Embodiment 28, wherein the average plasma concentration of GBPA in the subject is selected from the group consisting of about 0.09 μM to about 0.10 μM, 0.10 μM to about 0.11 μM, about 0.11 μM to about 0.12 μM, and about 0.12 μM to about 0.13 μM.

Embodiment 30 provides the method of any one of Embodiments 1-29, wherein the administration affords a maximal plasma concentration of GBPA in the subject is about 140 ng/ml to about 160 ng/ml.

Embodiment 31 provides the method of Embodiment 30, wherein the maximal plasma concentration of GBPA in the subject is selected from the group consisting of about 140 ng/ml to about 150 ng/ml and about 150 ng/ml to about 160 ng/ml.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

1. A method of treating, ameliorating, preventing, shortening duration, or reversing at least one symptom or complication in a human subject with a coronavirus infection,
the method comprising administering to the subject a dose of about 645 mg of camostat free base, or an equimolar amount of a pharmaceutically acceptable salt or solvate thereof.

2. A method of preventing or reducing the occurrence of long COVID, or a symptom or complication thereof, in a human subject with a SARS-CoV-2 infection; hospitalization of a human subject with a SARS-CoV-2 infection; or death in a human subject with a SARS-CoV-2 infection;
the method comprising administering to the subject a dose of about 645 mg of camostat free base, or an equimolar amount of a pharmaceutically acceptable salt or solvate thereof.

3. The method of claim 1, wherein the coronavirus is at least one of MERS-CoV, SARS-CoV, or SARS-CoV-2.

4. (canceled)

5. The method of claim 1, wherein the subject suffers from long COVID.

6. The method of claim 1, wherein the administering reverses, reduces, or prevents progression of the coronavirus infection, optionally wherein the progression of the coronavirus comprises hospitalization or death.

7. (canceled)

8. The method of claim 1, wherein at least one of the following applies:
(a) the administering reduces, reverses, or eliminates at least one symptom of the coronavirus infection, and
(b) the administering reduces recovery time for at least one symptom of the coronavirus infection.

9. (canceled)

10. The method of claim 1, wherein the at least one symptom is selected from the group consisting of abnormal, reduced, or eliminated sense of smell (e.g., anosmia), abnormal, reduced, or eliminated sense of taste (e.g., ageusia), runny nose, congested nose, sinus pressure, sneezing, scratchy or itchy throat, sore or painful throat, swollen throat, difficulty swallowing, teary or water eyes, sore or painful eyes, eyes sensitive to light, difficulty breathing, chest congestion, chest tightness, dry or hacking cough, wet or loose cough, frequent coughing, coughing mucus or phlegm, lack of appetite, gastrointestinal discomfort (i.e., stomachache), vomiting, diarrhea, headache, head congestion, dizziness, lightheadedness, nausea, dyspnea, myalgia, fever, excessive sleeping, difficulty sleeping, body aches or pains, fatigue, chills or shivering, feeling cold, feeling hot, sweating, and discomfort.

11-12. (canceled)

13. The method of claim 8, wherein the recovery time is reduced by at least about 1 to about 10 days as compared to a control human subject who is not administered the daily dose of about 645 mg of camostat free base, or an equimolar amount of a pharmaceutically acceptable salt or solvate thereof, optionally wherein the recovery time is reduced by about 5 days as compared to a control human subject who is not administered the daily dose of about 645 mg of camostat free base, or an equimolar amount of a pharmaceutically acceptable salt or solvate thereof.

14. (canceled)

15. The method of claim 1, wherein the administration to the subject is by at least one route selected from the group consisting of nasal, inhalational, topical, oral, buccal, rectal, pleural, peritoneal, vaginal, intramuscular, subcutaneous, transdermal, epidural, intratracheal, otic, intraocular, intrathecal, and intravenous routes.

16. (canceled)

17. The method of claim 1, wherein the administration of the dose to the subject is performed daily.

18. The method of claim 1, wherein the administration to the subject is performed for a period of 7 days.

19. The method of claim 17, wherein the daily administration comprises 800 mg of camostat mesylate.

20. The method of claim 17, wherein the daily administration comprises four equal doses of camostat free base, or a pharmaceutically acceptable salt or solvate thereof, optionally wherein each of the four equal doses comprises about 200 mg of camostat mesylate.

21. (canceled)

22. The method of claim 20, wherein each dose is administered within about 3, 4, 5, or 6 hours of the previous or following dose.

23-25. (canceled)

26. The method of claim 1, wherein the administration affords a trough plasma concentration of 4-(4-guanidinobenzoyloxy)phenylacetic acid (GBPA) in the subject of about 0.1 ng/ml to about 25 ng/ml.

27. The method of claim 26, wherein the trough plasma concentration of GBPA in the subject is selected from the group consisting of about 0.1 ng/ml to about 5 ng/ml, about 5 ng/ml to about 10 ng/ml, about 10 ng/ml to about 15 ng/ml, about 15 ng/ml to about 20 ng/ml, and about 20 ng/ml to about 25 ng/ml.

28. The method of claim 1, wherein the administration affords an average plasma concentration of GBPA in the subject of about 0.09 μM to about 0.13 μM.

29. The method of claim 28, wherein the average plasma concentration of GBPA in the subject is selected from the group consisting of about 0.09 μM to about 0.10 μM, 0.10 μM to about 0.11 about 0.11 μM to about 0.12 and about 0.12 μM to about 0.13 μM.

30. The method of claim 1, wherein the administration affords a maximal plasma concentration of GBPA in the subject is about 140 ng/ml to about 160 ng/ml.

31. The method of claim 30, wherein the maximal plasma concentration of GBPA in the subject is selected from the group consisting of about 140 ng/ml to about 150 ng/ml and about 150 ng/ml to about 160 ng/ml.
